(19) United States
(12) Patent Application Publication
(10) Pub. No.: US 2025/0186005 A1
(43) Pub. Date: Jun. 12, 2025

(54) MEDICAL IMAGE PROCESSING METHOD, MEDICAL IMAGE PROCESSING DEVICE, AND STORAGE MEDIUM STORING MEDICAL IMAGE PROCESSING PROGRAM

(71) Applicant: NIDEK CO., LTD., Gamagori-shi (JP)

(72) Inventor: Masanori NAKANO, Gamagori-shi (JP)

(21) Appl. No.: 18/970,913

(22) Filed: Dec. 6, 2024

(30) Foreign Application Priority Data
Dec. 7, 2023 (JP) 2023-207321

(51) Int. Cl. A61B 6/03; A61B 6/00; A61B 6/46

(52) U.S. Cl. CPC A61B 6/03; A61B 6/461; A61B 6/54

(57) ABSTRACT

A medical image processing device includes a control unit configured to perform: an image acquisition step of acquiring a front image and a tomographic image that are captured from a same living tissue of a same subject; an image display step of displaying the front image and the tomographic image on a display unit; and when an instruction for changing a display magnification of one of the front image and the tomographic image is input into the display unit, a display magnification change step of changing, in accordance with the input instruction, both a display magnification of the one of the front image and the tomographic image for which the instruction was given and a display magnification of an other of the front image and the tomographic image for which the instruction was not given.

### CROSS REFERENCE TO RELATED APPLICATION

This application is based on, and claims the benefit of priority from Japanese Patent Application No. 2023-207321 on Dec. 7, 2023. The entire disclosure of the above application is incorporated herein by reference.

### TECHNICAL FIELD

The present disclosure relates to a medial image processing method, a medical image processing device that is configured to process image data of a living tissue, and a storage medium storing a medical image processing program executed in the medical image processing device.

### BACKGROUND

Images of living tissues are useful for assisting users (e.g., medical professionals, etc.) in the treatment of patients. In recent years, technology for capturing tomographic images that extend in the depth direction of a living tissue has also been actively used. For example, an ophthalmic imaging device displays a tomographic image and a front image of a subject eye side by side on the same screen of a monitor.

### SUMMARY

Healthcare professionals often need useful information about a patient by comparing the front image and the tomographic image of the same living tissue. However, in the conventional technology, the user can change only the displaying mode of each of the front image and the tomographic image separately, so that a complicated operation may be required to properly compare the front image and the tomographic image.

One typical objective of the present disclosure is to provide a medical image processing method, a medical image processing device, and a medical image processing program that allow a user to easily and appropriately compare a front image and a tomographic image of a same living tissue.

A medical image processing method provided by a typical embodiment is a medical image processing method for processing image data of a living tissue, comprising: acquiring a front image and a tomographic image that are captured from a same living tissue of a same subject, wherein the front image is a two-dimensional image of the living tissue when viewed in a direction along an optical axis of imaging light, and the tomographic image is a two-dimensional image that spreads in a depth direction of the living tissue; displaying the front image and the tomographic image on a display unit; specifying one of the front image and the tomographic image that are displayed on the display unit by setting a reference position on the one of the front image and the tomographic image through an operation unit controlled by a user, wherein the reference position serves a reference for enlarging and minimizing an image displayed on the display unit; changing a display magnification of the specified one of the front image and the tomographic image in response to the user operating the operation unit; and synchronously changing a display magnification of an other of the front image and the tomographic image in accordance with a change in the display magnification of the specified one of the front image and the tomographic image.

A medical image processing device provided by a typical embodiment in the present disclosure is a medical image processing device that processes image data of a living tissue. The medical image processing device includes a control unit that is configured to perform: an image acquisition step of acquiring a front image and a tomographic image that are captured from a same living tissue of a same subject, wherein the front image is a two-dimensional image of the living tissue when viewed in a direction along an optical axis of imaging light, and the tomographic image is a two-dimensional image that spreads in a depth direction of the living tissue; an image display step of displaying the front image and the tomographic image on a display unit; and when an instruction for changing a display magnification of one of the front image and the tomographic image is input into the display unit, a display magnification change step of changing, in accordance with the input instruction, both a display magnification of the one of the front image and the tomographic image for which the instruction was given and a display magnification of an other of the front image and the tomographic image for which the instruction was not given.

A non-transitory, computer readable, storage medium storing provided by a typical embodiment in the present disclosure is a non-transitory, computer readable, storage medium storing a medical image processing program executed by a medical image processing device that processes image data of a living tissue, the medical image processing program, when executed by a control unit of the medical image processing device, causing the control unit to perform: an image acquisition step of acquiring a front image and a tomographic image that are captured from a same living tissue of a same subject, wherein the front image is a two-dimensional image of the living tissue when viewed in a direction along an optical axis of imaging light, and the tomographic image is a two-dimensional image that spreads in a depth direction of the living tissue; an image display step of displaying the front image and the tomographic image on a display unit; and when an instruction for changing a display magnification of one of the front image and the tomographic image is input into the display unit, a display magnification change step of changing, in accordance with the input instruction, both a display magnification of the one of the front image and the tomographic image for which the instruction was given and a display magnification of an other of the front image and the tomographic image for which the instruction was not given

According to the medical image processing method, the medical image processing device and the storage medium storing the medical image processing program according to the present disclosure, the user can easily and appropriately compare the front image and the tomographic image of the same living tissue.

### BRIEF DESCRIPTION OF DRAWINGS

FIG. 1 is a block diagram showing a schematic configuration of a medical image processing system 100.

FIG. 2 is an explanatory diagram for explaining a method implemented by an imaging device 1 in the present embodiment for capturing a three-dimensional image of a living tissue 50.

FIG. 3 is an explanatory diagram for explaining a state in which a three-dimensional image is being formed by a plurality of tomographic images 61.

FIG. 4 is a diagram illustrating one example of the three-dimensional image 60.

FIG. 5 is a diagram showing one example of a state in which a front image 70 and tomographic images 80X and 80Y are displayed on a monitor 47.

FIG. 6 is a flowchart of an image display process executed by the medical image processing device 40 according to the first embodiment.

FIG. 7 is a flowchart of a displaying-manner change process executed during the image display process according to the first embodiment.

FIG. 8 is a diagram illustrating one example of the front image 70 and the tomographic image 80X before enlarged and the front image 70 and the tomographic image 80X after enlarged in the first embodiment.

FIG. 9 is a flowchart of an image display process executed by a medical image processing device 40 according to the second embodiment.

FIG. 10 is a flowchart of a displaying-manner change process executed during the image display process according to the second embodiment.

FIG. 11 is a diagram illustrating one example of front images 70A, 70B, 70C and tomographic images 80XA, 80XB, 80XC before enlarged and the front images 70A, 70B, 70C and the tomographic images 80XA, 80XB, 80XC after enlarged in the second embodiment.

### DESCRIPTION OF EMBODIMENTS

### Overview

The medical image processing device exemplified in the present disclosure processes image data of living tissues. A control unit of the medical image processing device executes an image acquisition step, an image display step, and a magnification synchronization change step. In the image acquisition step, the control unit acquires a front image and a tomographic image on the same living tissue of the same subject. The front image is a two-dimensional image when the living tissue is viewed in a direction along an optical axis of an imaging light. The tomographic image is a two-dimensional image that extends in a depth direction of a living tissue. At the image display step, the control unit displays the front image and the tomographic image on a display unit. At the magnification synchronization change step, when an instruction for one of the front image and the tomographic image for changing a display magnification is input into the display unit, the control unit changes (i.e., enlarges or minimizes), in accordance with the input instruction, both a display magnification of the one of the front image and the tomographic image for which the display magnification change instruction was input and a display magnification of an other of the front image and the tomographic image for which the display magnification change instruction was not input.

According to the present disclosure, both the display magnification for the front image and the display magnification for the tomographic image are changed synchronously by inputting, into the medical image processing device, the display magnification change instruction for either the front image or the tomographic image that are displayed on the display unit. Thus, the user can easily and appropriately compare the front image and the tomographic image as compared to individually changing the magnification of the front image and the magnification of the tomographic image.

At the magnification synchronization change step, the control unit may change the display magnification of the front image and the display magnification of the tomographic image in display frames while maintaining a size and a shape of each of the display frames in which the front image and the tomographic image are respectively displayed. In this case, even if the display magnification of the front image and the display magnification of the tomographic image are changed, each image is displayed in the same display frame. Therefore, the user can appropriately check the image. However, the control unit may change the size or the shape of the display frame of at least one of the front image and the tomographic image according to the display magnification of the image. For example, in the display frame of the tomographic image, the size of the display frame in a depth direction may be changed according to the display magnification, while maintaining the size of the display frame in a direction intersecting the depth direction.

The front image may be an angiography front image captured by an OCT device. The OCT device can capture images of living tissues using the principle of optical coherence tomography. The angiography front image is a front image (Enface image in the present disclosure) generated by motion contrast data (i.e., OCT angography data). The motion contrast data is generated by processing a plurality of OCT signals acquired at different times at the same position on a living tissue. The motion contrast data includes information of the movement in a tissue (for example, the movement of blood flow in a blood vessel in a living tissue, etc.). Accordingly, by checking the angiography front image displayed on the display unit, a user can appropriately recognize the motion (for example, blood flow) of the living tissue from the front side of the tissue.

The tomographic image may be captured by an OCT device. As described above, the OCT device can capture images of living tissues using the principle of optical coherence tomography. Information of the motion contrast data may be superimposed on the tomographic image. As described above, the motion contrast data is generated by processing a plurality of OCT signals acquired at different times at the same position on a living tissue. The motion contrast data includes information of the movement in a tissue (for example, the movement of blood flow in a blood vessel in a living tissue, etc.). Therefore, the user can appropriately recognize the movement (for example, blood flow, etc.) in the living tissue on the tomographic image by checking the tomographic image on which the motion contrast data information is superimposed.

It should be noted that both the angiography front image and the tomographic image on which the motion contrast data information is superimposed (hereinafter, referred to as “MC superimposed tomographic image”) may be displayed on the display unit. In this case, the user can appropriately recognize information on movement such as blood flow in a living tissue from both the angiography front image and the MC superimposed tomographic image. For example, the state of the tissue for the portion that appears in the front image as a blood vessel can be checked on the tomographic image. Further, according to the present disclosure, the display magnifications of both the angiography front image and the MC superimposed tomographic image are changed synchronously by inputting only an instruction to change the display magnification for one of the images. Therefore, by comparing the angiography front image with the MC superimposed tomographic image in which the display magnifications are changed synchronously, the user can more easily and appropriately recognize the movement such as blood flow in a living tissue.

At the image acquisition step, the control unit may acquire both the front image and the tomographic image from data of the same three-dimensional image (for example, a three-dimensional image captured by an OCT device). In this case, since the relative positional relationship between the acquired front image and the acquired tomographic image is clarified, the condition of the living tissue may be appropriately acquired.

The three-dimensional image may be formed by arranging a plurality of two-dimensional B-scan images in a direction intersecting a plane direction in which each B-scan image extends. The B-scan image is a two-dimensional tomographic image taken by scanning with a spot of measurement light emitted by the OCT device. The B-scan image is a two-dimensional image that extends in a direction in which the spot of the measurement light is moved and in a depth direction (i.e., Z direction) along the optical axis of the measured light. In this case, the tomographic image displayed on the display unit may be at least one of the plurality of B-scan images constituting a three-dimensional image. Further, the control unit may extract a two-dimensional tomographic image that extends in the depth direction and in a direction intersecting each B-scan image from the three-dimensional image. Then, the control unit may display the extracted image on the display unit. The number of tomographic images displayed together with the front image may be one or multiple.

However, the method for capturing images by the OCT device is not necessarily limited to a method for scanning with the spot of the measurement light. For example, an irradiation optical system of the OCT device may simultaneously emit measurement light onto a two-dimensional region in the living tissue of the subject. In this case, the photodetector may be a two-dimensional photodetector that detects an interference signal in the two-dimensional region in the tissue. That is, the OCT device may capture a three-dimensional image according to the principle of so-called full-field OCT (FF-OCT). Further, the OCT device may simultaneously emit the measurement light on an irradiation line extending in a one-dimensional direction in the tissue, while scanning by moving the measurement light in a direction intersecting the irradiation line. In this case, the photodetector may be a one-dimensional photodetector (for example, a line sensor) or a two-dimensional photodetector. That is, the OCT device may capture a three-dimensional image according to the principle of so-called line-field OCT (LF-OCT).

Further, the control unit may change, in a region of the three-dimensional image, the position at which the tomographic image to be displayed on the display unit is extracted according to the position at which the front image is enlarged. For example, the control unit may maintain the extraction position for the tomographic image in the region of the three-dimensional image at a specific position (e.g., a center position) in the display frame of the front image displayed on the display unit. In this case, since the relative positional relationship between the acquired front image and the tomographic image is appropriately changed, the condition of the living tissue can be appropriately acquired.

However, the front image and the tomographic image may be images that are captured separately. Further, the imaging device for capturing the front image and the imaging device for capturing the tomographic image may be different devices.

Further, the front image displayed by the display unit is not necessarily limited to the angiography front image. For example, when an OCT device is used as the imaging device for capturing the front image, the front image may be an Enface image generated based on three-dimensional OCT data (may not necessarily be motion contrast data). Data of the Enface image may be, for example, integrated image data obtained by integrating luminance values in a depth direction (Z direction) at each position in X-Y direction, integrated values of spectral data at each position in X-Y direction, luminance data at each position in X-Y direction with a certain depth, and luminance data at each position in X-Y direction in any layer of a living tissue (for example, the retinal surface layer, etc.). The front image may be a thickness map indicating a two-dimensional distribution of the thickness of a particular layer in a living tissue. Further, by inputting a three-dimensional image into a mathematical model trained by a machine learning algorithm, the control unit may acquire a probability distribution for identifying a specific structure (for example, at least one of a layer and a boundary) in the living tissue appearing in the three-dimensional image. The control unit may acquire, as a front image, a deviation degree map showing a two-dimensional distribution of the degree of deviation of the acquired probability distribution with respect to the probability distribution that will be generated when the target structure is accurately identified. The front image may be taken by a device different from the OCT device (for example, at least one of a fundus camera, a scanning laser ophthalmoscope (SLO), an infrared camera, and the like).

The tomographic image displayed on the display unit is not necessarily limited to a tomographic image captured by the OCT device. For example, a tomographic image taken by an MRI (magnetic resonance imaging) device, a CT (computed tomography) device, or the like may be displayed on the display unit.

At the magnification synchronization change step, the control unit may change both the display magnification of the front image and the display magnification of the tomographic image while synchronizing a display range for the tomographic image in the display unit in an extending direction of the tomographic image that is a direction perpendicular to the depth direction and a display range for the front image in the display unit in the extending direction. In this case, even if the display magnifications of both the front image and the tomographic image are changed synchronously, the display range in the extending direction for the front image matches the display range in the extending direction for the tomographic image. Thus, the user can easily compare the front image with the tomographic image on the target site.

The control unit may perform a layer/boundary acquisition step for acquiring the position of a specific layer or boundary among the living tissue appearing in the tomographic image displayed on the display unit. At the magnification synchronization change step, when an instruction for the front image for changing the display magnification, the control unit may change the display magnification of the tomographic image while maintaining an average position, in the depth direction, of at least one of a specific layer and a specific boundary (hereinafter, referred to as a “layer/boundary”) in the tomographic image at a target position in the depth direction (e.g., a predetermined position or a position calculated according to the display magnification). In this case, regardless of the display magnification of the tomographic image, the specific layer/boundary is likely to be captured close around a predetermined position in the depth direction (for example, near the center or the position immediately before changing the display magnification, etc.). Therefore, even when the display magnification of the front image and the display magnification of the tomographic image are changed, the user can appropriately observe the specific layer/boundary in the tomographic image.

Note that, when the display magnification of the tomographic image is changed, the target position to maintain a position of the specific layer/boundary in the depth direction can be appropriately selected. For example, the control unit may change the display magnification of the tomographic image while maintaining an average position of the specific layer/boundary in the depth direction at the center of the depth direction in the display range in the image. Alternatively, the control unit may change the display magnification of the tomographic image while maintaining the average position of the specific layer/boundary in the depth direction at the position of the specific layer/boundary immediately before changing the display magnification. Yet alternatively, the control unit may specify a range in the depth direction (hereinafter, referred to as a “slab”) of the layer/boundary for which the Enface image described above is generated. Then, the control unit may change the display magnification of the tomographic image by setting a predetermined position (for example, the center position) in the specified slab in the depth direction as the center for changing the display magnification. The slab can be identified by the boundary on the surface side and the boundary on the deep side of the range within which the Enface image is generated. The boundary for specifying the slab may be a layer in the living body that actually appears in the image or may be a position that is offset from the layer of the living body a predetermined distance in the depth direction. By changing the display magnification of the tomographic image based on the slab, the specific layer/boundary can be observed more appropriately.

Further, at the magnification synchronization change step, the control unit may change the display magnification of the tomographic image only in the extending direction intersecting the depth direction synchronously with the display magnification of the front image without changing the display magnification of the tomographic image in the depth direction. In this case, even if the tomographic image is enlarged, at least one of the layer and the boundary (hereinafter, simply referred to as a “layer/boundary”) is less likely to be displayed at a position outside of the display range for the tomographic image. Therefore, the user can more appropriately observe the layer/boundary of interest on the tomographic image. Further, at the synchronization magnification change step, the control unit may change the display magnifications of both the images while maintaining both the aspect ratio of the front image and the aspect ratio of the tomographic image.

The control unit may acquire the front image, a first tomographic image, and a second tomographic image at the image acquisition step. The first tomographic image is a tomographic image that extends in the depth direction and a first extending direction (for example, X direction, etc.), which is one of directions that perpendicularly intersect the depth direction. The second tomographic image is a tomographic image that extends in the depth direction and a second extending direction (for example, Y direction, etc.), which is one of directions that perpendicularly intersect the depth direction. The first and second extending directions are different from each other. The control unit may display the front image, the first tomographic image, and the second tomographic image on the display unit at the image display step. At the magnification synchronization change step, when a display magnification change instruction for one of the plurality of images displayed on the display unit is input, the control unit may synchronously change the display magnification of the front image, the display magnification of the first tomographic image, and the display magnification of the second tomographic image. In this case, the display magnifications for the plurality of images can be synchronously changed by simply inputting, by the user, the display magnification change instruction for only one of the plurality of images while checking all the front image, the first tomographic image, and the second tomographic image. Therefore, it is possible to compare the multiple images easily and appropriately.

For example, the control unit may change the display magnification of the first tomographic image and the display magnification of the second tomographic image while synchronizing the display range of the first tomographic image in the depth direction and the display range of the second tomographic image in the depth direction with each other. The control unit may change the display magnification of the first tomographic image and the display magnification of the front image while synchronizing the display range of the first tomographic image in the first extending direction and the display range of the front image in the first extending direction with each other. The control unit may change the display magnification of the second tomographic image and the display magnification of the front image while synchronizing the display range of the second tomographic image in the second extending direction and the display range of the front image in the second extending direction with each other. In this case, the display magnification of each image is changed while the display range of the front image, the display range of the first tomographic image, and the display range of the second tomographic image are appropriately synchronized with each other.

The control unit may further execute a first reference position setting step and a second reference position setting step. At the first reference position setting step, the control unit sets a first reference position that serves as a reference for enlarging and minimizing the first tomographic image in the first extending direction (for example, serving as the center for enlarging/minimizing in the first extending direction, etc.). At the second reference position setting step, the control unit sets a second reference position that serves as a reference for enlarging and minimizing the second tomographic image in the second extending direction (for example, serving as the center for enlarging/minimizing in the second extending direction, etc.). At the magnification synchronization change step, the control unit sets the first reference position as a reference for enlarging and minimizing the first tomographic image (for example, the center for enlargement and minimization, etc.), and the second reference position as a reference for enlarging and minimizing the second tomographic image (for example, the center for enlargement and minimization, etc.) to synchronously change the display magnification of the first tomographic image and the display magnification of the second tomographic image. In this case, the display magnification of the first tomographic image and the display magnification of the second tomographic image are changed synchronously based on the reference position set for each of the first tomographic image and the second tomographic image. Thus, the user can easily check an image area in an appropriate range based on the set reference position in each of the first and second tomographic images. Note that the control unit may synchronously change the display magnification of the front image, the display magnification of the first tomographic image, and the display magnification of the second tomographic image by setting the first reference position as a reference for enlarging and minimizing the front image in the first extending direction and the second reference position as a reference for enlarging and minimizing the front image in the second extending direction. In this case, the display magnification of each of the plurality of the images can be changed appropriately and synchronously.

A specific method for setting the first reference position and the second reference position can be appropriately selected. For example, the control unit may acquire the front image, the first tomographic image, and the second tomographic image from the common three-dimensional image at the image acquisition step. The control unit may automatically set the first reference position at a position in the three-dimensional image in the first extending direction where the second tomographic image is extracted. The control unit may automatically set the second reference position at a position in the three-dimensional image in the second extending direction where the first tomographic image is extracted. In this case, the display magnification of one of the tomographic images is changed based on the position at which the other of the tomographic images is extracted. Therefore, the user can easily compare the plurality of images.

Further, the control unit may set at least one of the first reference position and the second reference position in response to an instruction input by a user. In this case, the user can change the display magnification of at least one of the first tomographic image and the second tomographic image based on the desired position.

The control unit may further execute a reference position display step for displaying the first reference position and the second reference position on the front image displayed on the display unit. In this case, the user can recognize both the first reference position for the first tomographic image and the second reference position for the second tomographic image on the front image. Therefore, the user can easily and appropriately recognize the first reference position and the second reference position, and input an instruction or the like for changing the display magnification of the image into the medical image processing device.

For example, the control unit may set the first reference position and the second reference position by displaying a first reference line that extends in the second extending direction and passes through the first reference position and a second reference line that extends in the first extending direction and passes through the second reference position. In this case, the user can better recognize the positional relationship between the first reference position and the second reference position.

Further, in addition to the lines on the front image, the control unit may further display a reference line that passes through the first reference position and extends in the depth direction on the first tomographic image. The control unit may display a reference line that passes through the second reference position and extends in the depth direction on the second tomographic image. In this case, the user can recognize the first and second reference positions in both the front image and the tomographic image.

The user may specify the first reference position by inputting an instruction for moving at least one of the first reference line on the front image and the reference line on the first tomographic image. Similarly, the user may specify the second reference position by inputting an instruction for moving at least one of the second reference line on the front image and the reference line on the second tomographic image. In this case, the user can easily and appropriately specify at least one of the first reference position and the second reference position.

However, it is also possible to change the method for displaying the first reference position and the second reference position. For example, the control unit may display, on the front image, only a cross point between a virtual first reference line that extends in the second extending direction and passes through the first reference position and a virtual second reference line that extends in the first extending direction and passes through the second reference position. In this case, the user can recognize both the first reference position and the second reference position simply by checking the position of the cross point displayed on the front image. Further, as will be described in detail later, the control unit may display a cursor moving in a display area of the display unit in response to an operation by a user. The control unit may set at least one of the first reference position and the second reference position based on the position of the cursor superimposed on the image. For example, when the cursor is displayed on the front image, the control unit may set the first reference position and the second reference position on the front image at the position at which the cursor is located. When the cursor is displayed on the first tomographic image, the control unit may set the first reference position on the first tomographic image at the position where the cursor is located. When the cursor is displayed on the second tomographic image, the control unit may set the second reference position on the second tomographic image at the position where the cursor is located.

Further, a specific method for synchronously changing the display magnification of the front image and the display magnification of the tomographic image can also be appropriately selected. For example, the control unit may synchronize the display magnification of the front image and the display magnification of the tomographic image based on imaging information of each of the front image and the tomographic image (for example, the imaging angle of the front image and the tomographic image, the number of scanning points, resolution, the model of the imaging device, and the axial length of the subject eye when the living tissue is the eye).

At the magnification synchronization change step, the control unit may receive an instruction for specifying, within the display area of at least one of the front image and the tomographic image, a reference position that serves as a reference for enlarging and minimizing. The control unit may perform an enlarging process or a minimizing process on at least one of the front image and the tomographic image based on the specified reference position as a center. In this case, the user can enlarge or minimize the image based on the specified reference position as a center by specifying, as the reference position, an appropriate position within the display range of at least one of the front image and the tomographic image. Thus, the user can easily compare the front image and the tomographic image.

Note that a specific method for specifying, by a user, the reference position can be appropriately selected. For example, the control unit may display, on the display unit, a cursor that moves in the display area in response to an operation such as a mouse. The control unit may set, as a reference position, a position of the cursor at that time in the display range of the front image or the tomographic image. Further, the reference position may be specified in the display range of the front image or the tomographic image by operating a touch panel or the like.

However, the method for setting the reference position may be changed. For example, the control unit may automatically set the reference position at the center position of at least one of the front image and the tomographic image. In this case, the image is enlarged and minimized about the center of the image. Further, when a tomographic image extending in a first direction and a tomographic image extending in a second direction different from the first direction are displayed together, the control unit may automatically set the reference position at the cross position where the tomographic image extending in the first direction and the tomographic image extending in the second direction intersect. In this case, the reference position can be set to an appropriate position based on the displayed tomographic image.

When an instruction for returning at least one of the display magnification and the display position of the image to an initial state is received, the control unit may further execute a display reset step for resetting at least one of the display magnification of the front image and the tomographic image and the display position to display within the display frame of the display unit to the state at the time of starting the display. In this case, the user can return at least one of the display magnification and the display position of the front image and the tomographic image back to the state at the time of starting the display with a simple operation.

When a display magnification change instruction is input into the display unit for one of the front image and the tomographic image, the control unit may execute a magnification single change step for enlarging or minimizing, in accordance with the input instruction, a display magnification on the one of the front image and the tomographic image for which the display magnification change instruction was input. The control unit may execute either the magnification synchronization change step or the magnification single change step according to an instruction input by the user. In this case, the user can appropriately use the magnification synchronization change step and the magnification single change step according to various situations. Thus, the user can better compare the two images.

The specific method for inputting an instruction to change the display magnification can be appropriately selected. For example, the user may input the display magnification change instruction into the medical image processing device by executing at least one of the following: mouse operation, key operation, touch panel operation, button operation displayed on the display unit, or the like.

At the image acquisition step, the control unit may acquire a plurality of image sets each of which is a set of the front image and the tomographic image captured as to the same living tissue of the same subject. The control unit may display the plurality of image sets on the display unit at the image display step. When the display magnification change instruction for one of a plurality of front images and a plurality of tomographic images is input into the display unit at the magnification synchronization change step, the control unit may change the display magnification of each of the plurality of front images and the plurality of tomographic images included in the plurality of image sets.

In this case, the user can appropriately recognize the condition of the living tissue by comparing the plurality of image sets taken from the same subject living tissue. Further, when the display magnification change instruction is input into the display unit for one of the plurality of front images and the plurality of tomographic images, not only the display magnification of the image for which the display magnification change instruction was input, but also the display magnifications of other images (the front images and the tomographic images) that are displayed on the display unit are changed in accordance with the input instruction. Thus, the user can compare the plurality of images (the front images and the tomographic images) more easily and appropriately as compare to individually changing the magnification of each of the plurality of images.

Note that the timing of imaging each of the plurality of image sets may be different from each other. In this case, the user can easily and appropriately observe (that is, follow-up) changes in the living tissues of the subject over time.

At the image display step, the control unit may perform alignment of the plurality of front images included in the plurality of image sets, and display, on the display unit, the plurality of front images that have been aligned with each other. In this case, the user can appropriately compare living tissues appearing in each of the plurality of front images at the same position. Therefore, the state of the living tissue can be appropriately recognized.

However, if the positions of the living tissues appearing in the plurality of front images already match, the alignment process for the plurality of front images may be omitted.

At the magnification synchronization change step, the control unit may set the reference position for enlargement and minimization at the same position in the display range of each of the plurality of front images, and may execute the enlarging process or the minimizing process on the plurality of front images based on the set reference position. The control unit may set the reference position for enlargement and minimization at the same position in the display range of each of the plurality of tomographic images, and executes an enlarging process or a minimizing process on the plurality of tomographic images based on the set reference position. In this case, the display magnifications of the plurality of front images are synchronously changed while aligning the display positions of the plurality of front images. Similarly, the display magnifications of the plurality of tomographic images are synchronously changed while aligning the display positions of the plurality of tomographic images. Thus, the user can easily and appropriately compare multiple images (i.e., the front images and the tomographic images).

At the image display step, the control unit may display, on the display unit, a plurality of tomographic images each having a common imaging position in the living tissue at which the images are captured. In this case, the user can appropriately compare multiple tomographic images each having the same position in the living tissue at which the images are captured. Therefore, the state of the living tissue can be appropriately recognized.

For example, the control unit may acquire a plurality of image sets by acquiring a front image and a tomographic image from each of a plurality of three-dimensional images that are captured at the same position in the same living tissue. In this case, the control unit may extract a plurality of tomographic images at the same position in the plurality of three-dimensional images. In this case, the imaging positions of the extracted plurality of tomographic images in the living tissue are common to each other. Note that the control unit may extract a plurality of tomographic images at the same position from the plurality of three-dimensional images after the plurality of three-dimensional images were aligned when viewed in a front direction. In this case, the imaging positions of the plurality of tomographic images in the living tissue can be close to each other. Therefore, the state of the living tissue can be appropriately recognized.

Further, when an instruction for one of the plurality of tomographic images for changing the extraction position in the three-dimensional image is input, the control unit may change the extraction positions for the tomographic images in the plurality of three-dimensional images to the same position. In this case, the user can change the extraction positions for the plurality of tomographic images at once by simply changing the extraction position of one of the plurality of tomographic images.

The technology according to the present disclosure can also be expressed as follows. A medical image processing method executed by a medical image processing device that processes image data of a living tissue, the medical image process method comprising: an image acquisition step of acquiring a front image and a tomographic image that are capture from a same living tissue of a same subject, wherein the front image is a two-dimensional image of the living tissue when viewed in a direction along an optical axis of imaging light, and the tomographic image is a two-dimensional image that spreads in a depth direction of the living tissue;
an image display step of displaying the front image and the tomographic image on a display unit; and
when an instruction for one of the front image and the tomographic image for changing a display magnification is input into the display unit, a display magnification change step of changing, in accordance with the input instruction, both a display magnification of the one of the front image and the tomographic image for which the instruction was given and a display magnification of an other of the front image of the tomographic image for which the instruction was not given.

### Embodiment

Hereinafter, one of exemplary embodiments according to the present disclosure will be described. In the present embodiment, image data of a fundus tissue of a subject eye E captured by an OCT device is processed. The OCT device can capture images of living tissues using the principle of optical coherence tomography. However, the images processed by the technique of the present disclosure may be images of a living tissue other than fundus tissue. For example, the processing target image may be an image of a living tissue other than the fundus of the subject eye E (for example, the anterior segment of the eye), or an image of a living tissue other than the subject eye E (for example, skin, digestive organs, or brain, etc.). Further, as described above, an imaging device for capturing images to be processed is not necessarily limited to the OCT device.

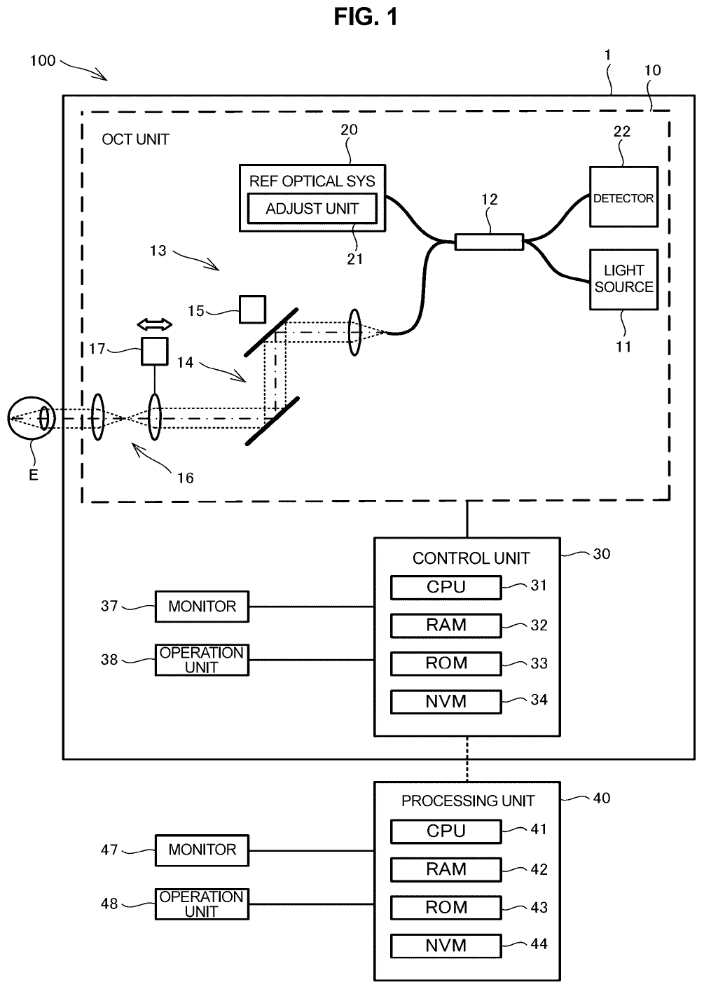

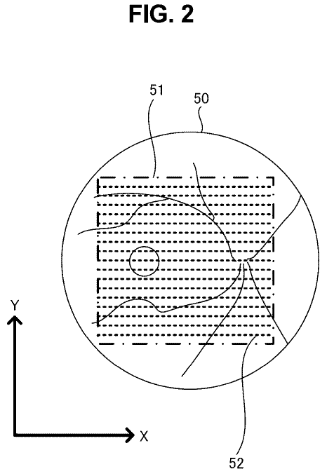

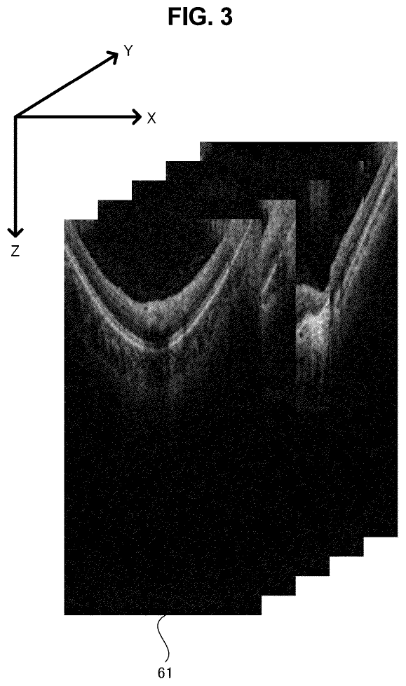

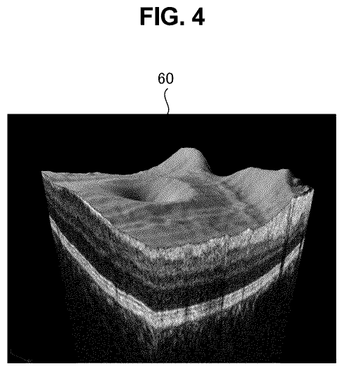

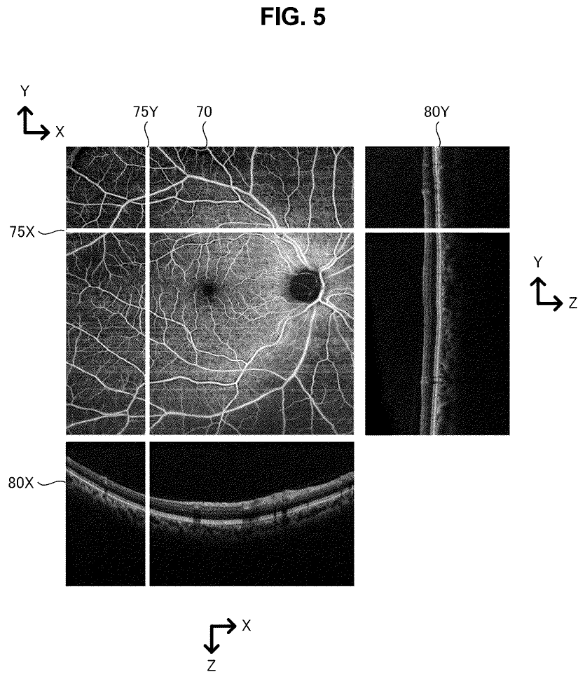

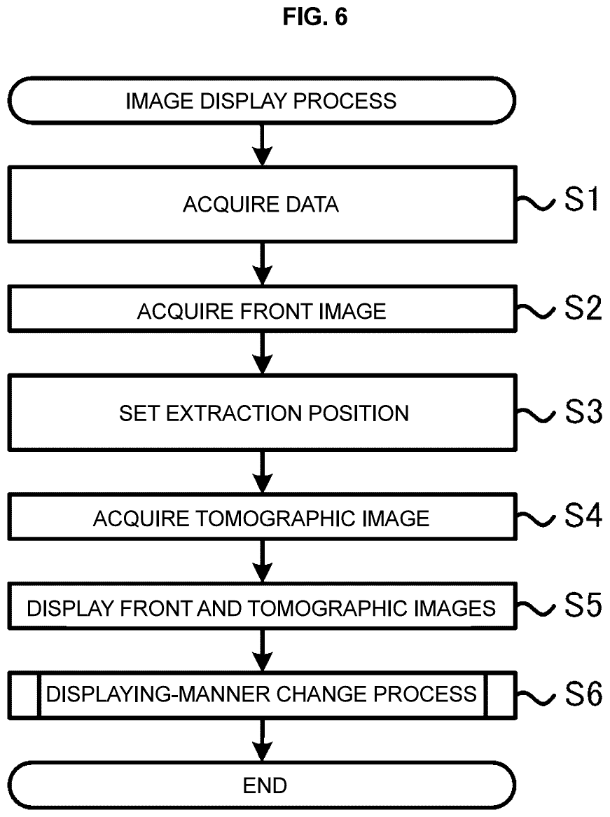

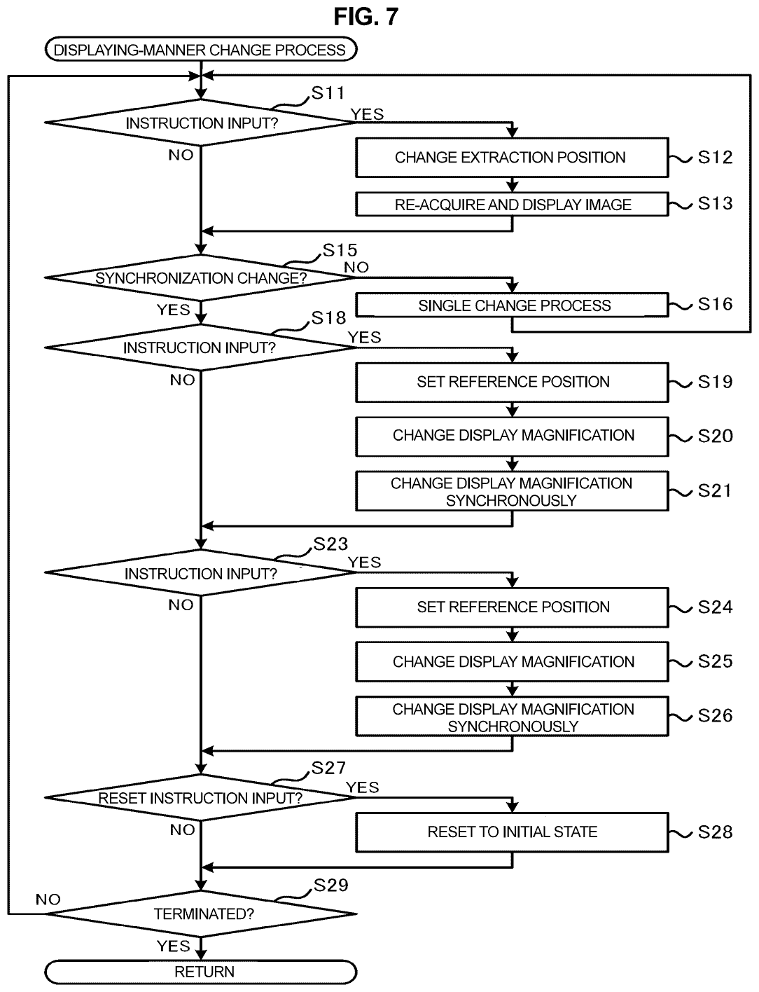

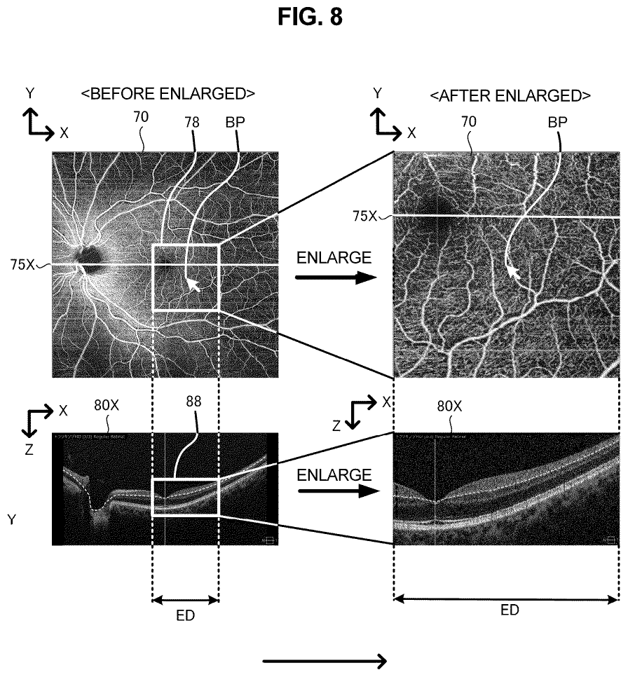

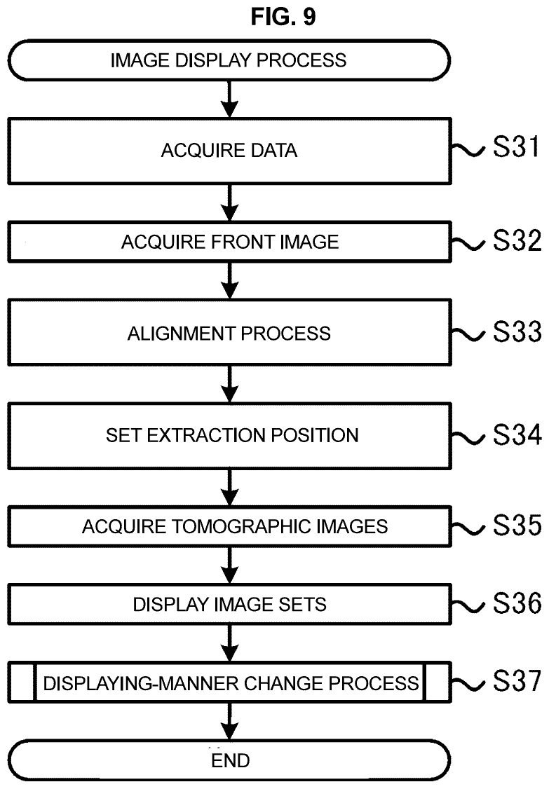

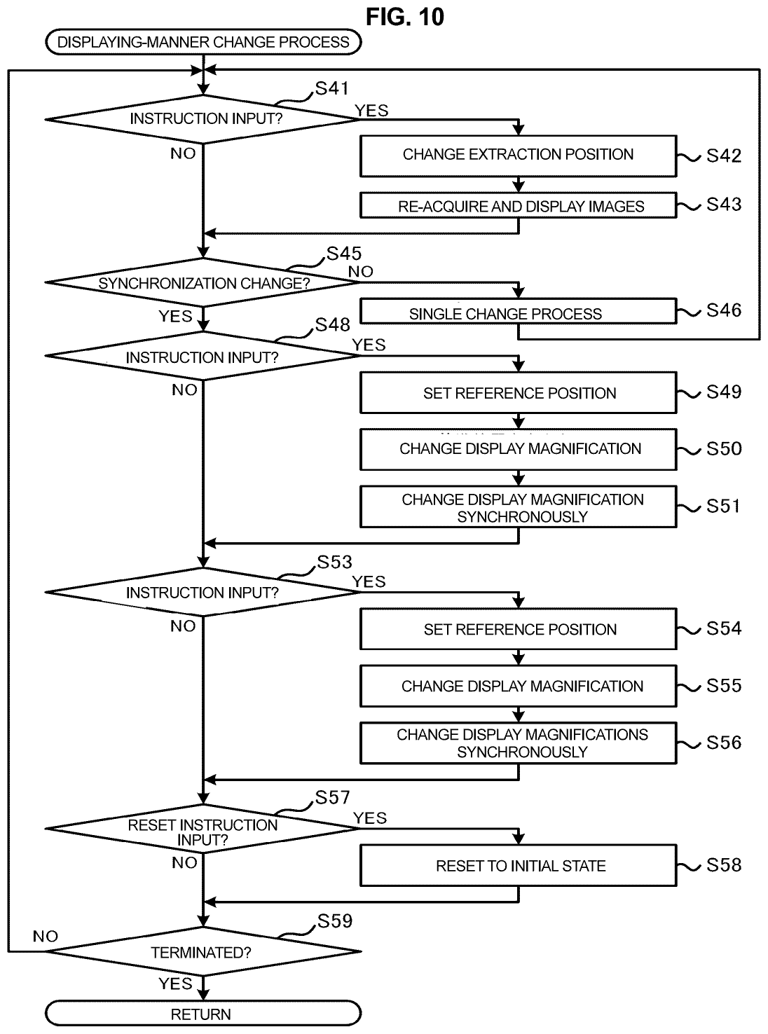

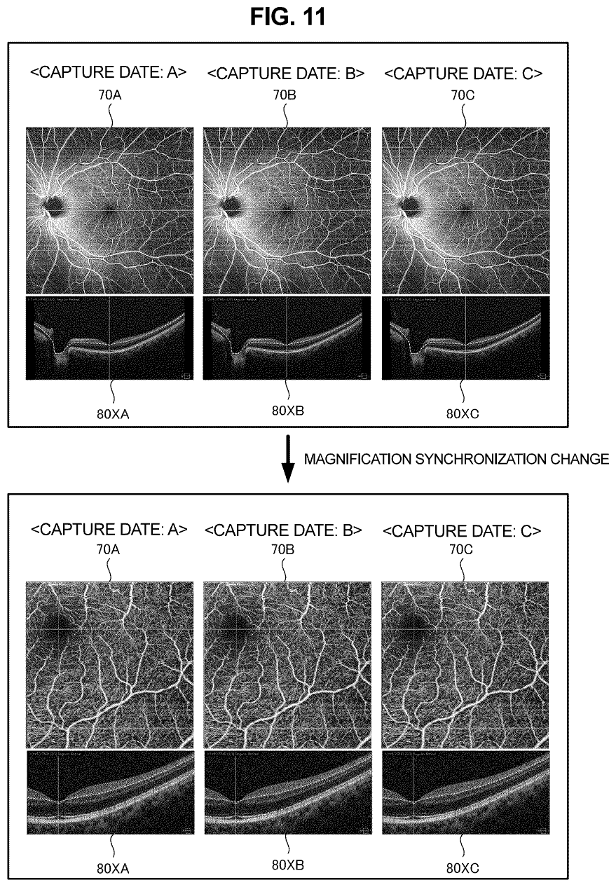

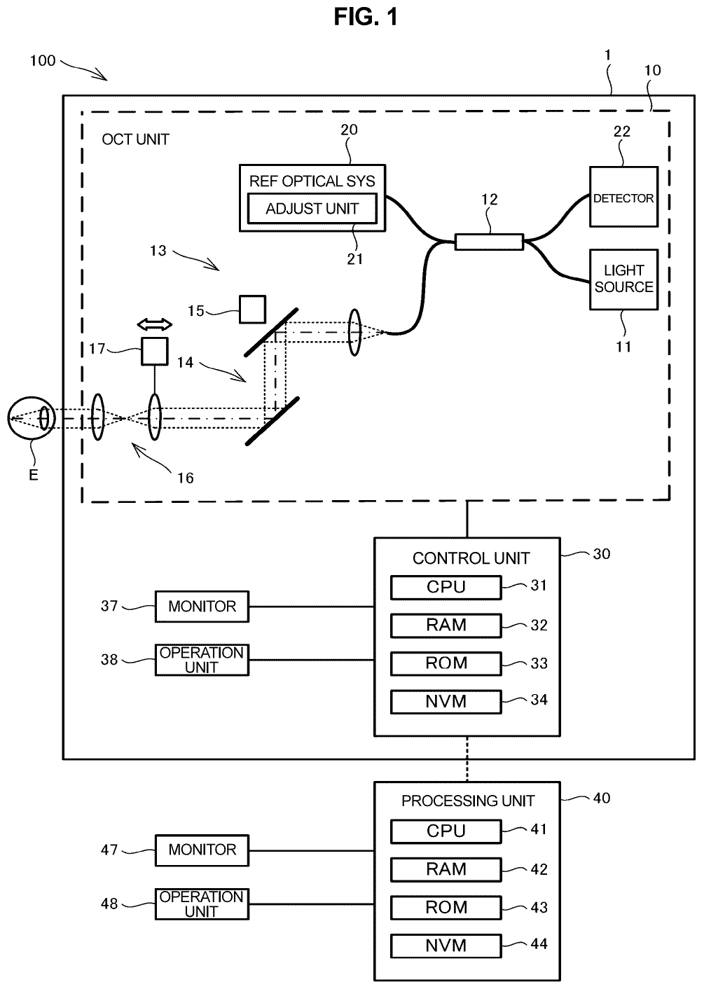

With reference to FIG. 1, a schematic configuration of a medical image processing system 100 in the present embodiment will be described. The medical image processing system 100 in the present embodiment includes an imaging device 1 and a medical image processing device 40. The imaging device (an OCT device in the present embodiment) 1 captures an image of a living tissue (a three-dimensional image in the present embodiment) by detecting light reflected by a living tissue. The medical image processing device 40 executes processing of image data captured by the imaging device 1 (for example, display control processing of images on a monitor 47, etc.). A PC is used in the medical image processing device 40 in the present embodiment. However, the device that may serve as the medical image processing device 40 is not necessarily limited to the PC. For example, the imaging device (the OCT device) 1 or the like may serve as the medical image processing device 40. When the imaging device 1 serves as the medical image processing device 40, the imaging device 1 can appropriately process captured images while capturing images of a living tissue. Further, a mobile terminal device such as a tablet terminal device or a smartphone may serve as the medical image processing device 40. The control units of multiple devices (for example, the CPU of the PC and the CPU 31 of the imaging device 1 may cooperatively perform various processes.

The configuration of the imaging device 1 in the present embodiment will be described. The imaging device (the OCT device) 1 includes an OCT unit 10 and a control unit 30. The OCT unit 10 includes an OCT light source 11, a coupler (light divider) 12, a measurement optical system 13, a reference optical system 20, and a photodetector 22.

The OCT light source 11 emits light (OCT light) for acquiring image data. The coupler 12 divides the OCT light emitted from the OCT light source 11 into a measurement light and a reference light. Further, the coupler 12 of the present embodiment combines the measurement light reflected by the living tissue (the fundus of the subject eye E in the present embodiment) and the reference light generated by the reference optical system 20 to have the measurement light and the reference light interfere with each other. That is, the coupler 12 of the present embodiment also serves as both a branching optical element that branches the OCT light into the measurement light and the reference light and a combined wave optical element that combines the reflected light of the measurement light and the reference light. The configuration of at least one of the branching optical element and the combined wave optical element may be changed. For example, an element other than the coupler (for example, a circulator, a beam splitter, or the like) may be used.

The measurement optical system 13 guides the measurement light divided by the coupler 12 to the subject eye, and returns the measurement light reflected by the living tissue to the coupler 12. The measurement optical system 13 includes a scanning unit (scanner) 14, an irradiation optical system 16, and a focus adjustment unit 17. The scanning unit 14 is configured to scan by moving a spot of the measurement light in a two-dimensional direction intersecting the optical axis of the measurement light by a driving unit 15. In the present embodiment, two galvanometer mirrors capable of deflecting the measurement light in different directions from each other are used as the scanning unit 14. However, another device (for example, at least one of a polygon mirror, resonant scanner, acousto-optic element, or the like) that deflects light may be used as the scanning unit 14. The irradiation optical system 16 is located on a downstream side of the scanning unit 14 in an optical path (that is, the side to the subject), and irradiates the living tissue with the measurement light. The focus adjustment unit 17 adjusts the focus of the measurement light by moving an optical element (for example, a lens) included in the irradiation optical system 16 in a direction along the optical axis of the measurement light.

The reference optical system 20 generates a reference light and returns the light to the coupler 12. The reference optical system 20 in the present embodiment generates the reference light by reflecting, by a reflective optical system (e.g., a reference mirror), the reference light divided by the coupler 12. However, the configuration of the reference optical system 20 may also be changed. For example, the reference optical system 20 may transmit light incident from the coupler 12 without reflecting the light and may return the light to the coupler 12. The reference optical system 20 includes an optical path length difference adjustment unit 21 that changes an optical path length difference between the measurement light and the reference light. In the present embodiment, the optical path length difference is changed by moving the reference mirror in the optical axis direction. The means for changing the optical path length difference may be provided in the optical path in the measurement optical system 13.

The photodetector 22 detects an interference signal by receiving the interference light of the measurement light and the reference light that are generated by the coupler 12. In this embodiment, the principle of Fourier domain OCT is used. In the Fourier domain OCT, the spectral intensity (the spectral interference signal) of the interference light is detected by the photodetector 22, and a complex OCT signal is acquired by the Fourier transform for the spectral intensity data. As an example of Fourier domain OCT, Spectral-domain-OCT (SD-OCT), Swept-source-OCT (SS-OCT), and the like can be used. Further, for example, it is possible to use Time-domain-OCT (TD-OCT) and the like.

Further, in the present embodiment, three-dimensional image data is acquired by scanning a spot of the measurement light in a two-dimensional region by the scanning unit 14. However, the principle of acquiring data for three-dimensional images may be changed. For example, three-dimensional image data may be acquired by the principle of line field OCT (hereinafter, referred to as “LF-OCT”). In LF-OCT, the measurement light is simultaneously emitted along an irradiation line extending in one-dimensional direction in a tissue, and the reflected light of the measurement light and the interference light of the reference light are received by a one-dimensional photodetector (for example, a line sensor) or a two-dimensional photodetector. In a two-dimensional measurement area, three-dimensional OCT data is acquired by scanning the measurement light in a direction intersecting the irradiation line. Further, the three-dimensional image data may be acquired by the principle of full-field OCT. In the full-field OCT, a two-dimensional region on a tissue of the subject is simultaneously irradiated with measurement light, and the interference light is received by a two-dimensional photodetector.

Further, based on the acquired three-dimensional OCT data, the imaging device 1 acquires (generates) an Enface image, which is a two-dimensional front image when the tissue is viewed in a direction (a frontal direction) along the optical axis of the measurement light (an imaging light). When acquiring an Enface image in real time, the acquired Enface image may also be used as an image for observing the living tissue from a front side (i.e., a frontal observation image). Data of the Enface image may be, for example, integrated image data obtained by integrating luminance values in a depth direction (Z direction) at each position in X-Y direction, integrated values of spectral data at each position in X-Y direction, luminance data at each position in X-Y direction with a certain depth, and luminance data at each position in X-Y direction in any layer of a retina (for example, the retinal surface layer). Further, the imaging device 1 in the present embodiment can also generate an Enface image from motion contrast data. The motion contrast data is data obtained by processing a plurality of OCT signals acquired at different times at the same position on a living tissue. The motion contrast data includes information of the movement of a living tissue (for example, the movement of blood flow in a blood vessel in a living tissue, etc.). In the present embodiment, an angiography front image (a blood vessel image) is generated that is an image indicating the position of the blood vessel in a specific layer by generating an Enface image of the specific layer based on the motion contrast data.

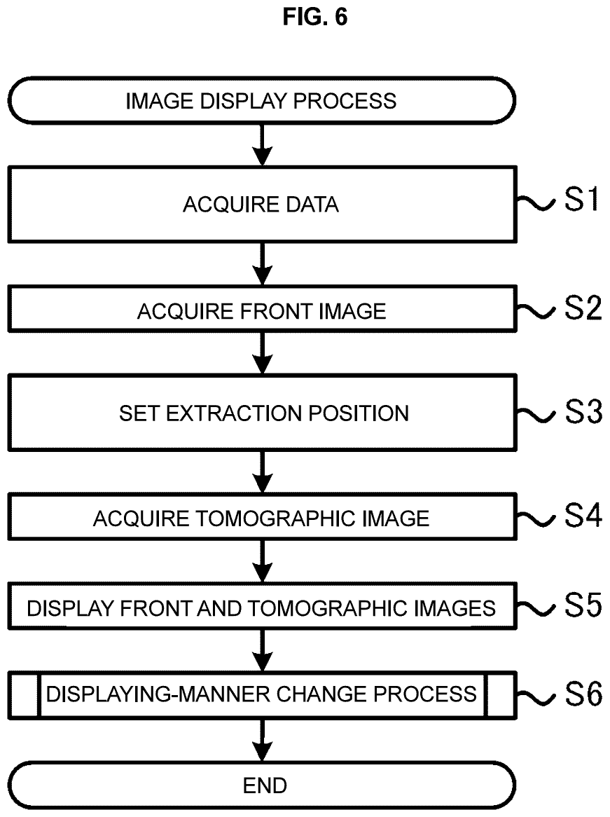

The control unit 30 performs various controls on the imaging device 1. The control unit 30 includes a CPU 31, a RAM 32, a ROM 33, and a non-volatile memory (NVM) 34. The CPU 31 is a controller that performs various controls. The RAM 32 temporarily stores various information. The ROM 33 stores programs to be executed by the CPU 31, various initial values, and the like. The NVM34 is a non-transient storage medium that is configured to keep storage contents even when a power supply is interrupted. When the imaging device 1 serves as a medical image processing device, a medical image processing program for executing an image display process described later (see FIGS. 6, 7, 9, and 10) may be stored in the NVM 34 or the like.

The monitor 37 and an operation unit 38 are connected to the control unit 30. The monitor 37 is one example of a display unit for displaying various images. The operation unit 38 is operated by a user in order for the user to input various operation instructions into the imaging device 1. For example, various devices such as a mouse, keyboard, touch panel, and foot switch can be used as the operation unit 38. Note that various operation instructions may be input into the imaging device 1 by inputting sound via a microphone.

A schematic configuration of the medical image processing device 40 will be described below. The medical image processing device 40 includes a CPU 41, a RAM 42, a ROM 43, and an NVM 44. A medical image processing program for performing an image display process (see FIGS. 6, 7, 9, and 10) described later may be stored in the NVM 44. Further, a monitor 47 and an operation unit 48 are connected to the medical image processing device 40. The monitor 47 is one example of a display unit for displaying various images. The operation unit 48 is operated by a user in order for the user to input various operation instructions into the medical image processing device 40. As the operation unit 48, various devices such as a mouse, a keyboard, and a touch panel can be used as with the operation unit 38 of the imaging device 1. Further, by inputting sound via a microphone, various operation instructions may be input into the medical image processing device 40.

The medical image processing device 40 acquires various data (for example, image data captured by the imaging device 1) from the imaging device 1. Various data may be acquired by, for example, at least one of wired communication, wireless communication, and a removable storage device (e.g., a USB memory), etc.

### Image

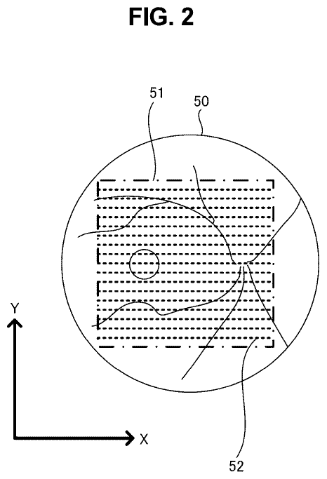

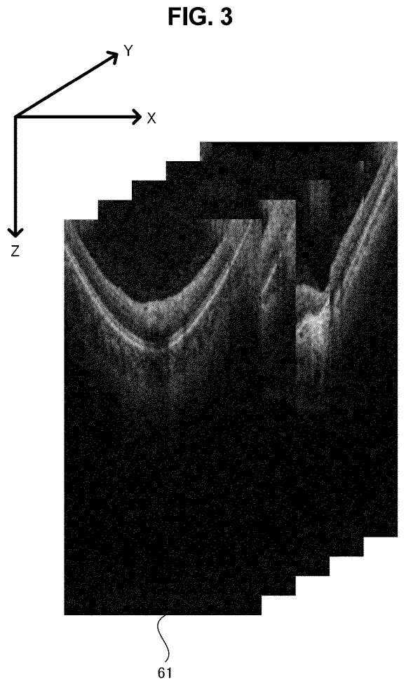

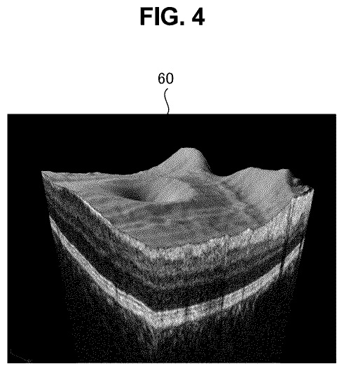

Referring to FIGS. 2 to 5, one example of an image to be processed by the medical image processing device 40 in the present embodiment will be described. As shown in FIG. 2, the imaging device 1 in the present embodiment scans the two-dimensional imaging region 51 in the living tissue 50 (in the example shown in FIG. 2, a fundus tissue) with a light (the measurement light). Specifically, the imaging device 1 in the present embodiment emits light on a scan line 52 extending in a predetermined direction in the imaging region 51, thereby imaging (i.e., capturing) a two-dimensional tomographic image (a B-scan image) 61 (see FIG. 3) extending in Z direction along the optical axis of light and in X direction perpendicular to Z direction. In the example shown in FIG. 3, Z direction is a direction perpendicular to the two-dimensional imaging region 51 (in the depth direction of the living tissue 50), and X direction is a direction in which the scan line 52 extend. Next, the imaging device 1 moves the position of the scan line 52 in Y direction on the imaging region 51 and repeats imaging (capturing) the tomographic image 61. Y direction is a direction that intersects both Z direction and X direction (perpendicularly intersecting in the present embodiment). As a result, a plurality of tomographic images 61 that each pass through each of the plurality of scan lines 52 and extend in the depth direction of the living tissue 50 are acquired. Then, as shown in FIG. 3, the plurality of tomographic images 61 are arranged in Y direction (in the direction of intersecting the imaging region of each tomographic image) to generate a three-dimensional image 60 (see FIG. 4) in the imaging region 51. That is, the image data captured by the imaging device 1 in the present embodiment is image data of a three-dimensional image that spreads in Z direction that is the depth direction of the living tissue and the two-dimensional X-Y direction that intersects Z direction.

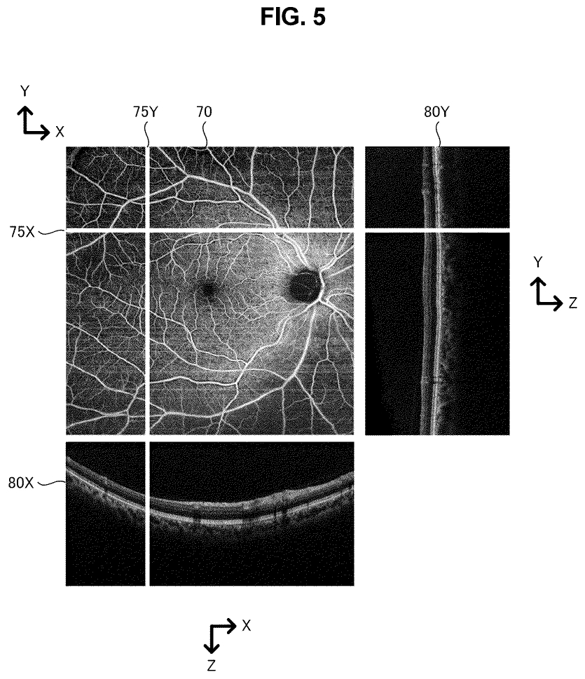

The medical image processing device 40 (in detail, the CPU 41) acquires both a front image 70 and a tomographic image 80 (see FIG. 5) from the data of the same three-dimensional image 60. The medical image processing device 40 displays the acquired front image 70 and the acquired tomographic image 80 on the monitor 47. In this case, since the relative positional relationship between the acquired front image 70 and the acquired tomographic image 80 is clarified, the condition (i.e., the status) of the living tissue can be appropriately acquired.

The medical image processing device 40 extracts a two-dimensional tomographic image 80 from the image area of the three-dimensional image 60 (see FIG. 4) and displays the extracted tomographic image 80 on the monitor 47. In the example shown in FIG. 5, in the image area of the front image 70, an extraction line 75X indicating the extraction position of the tomographic image 80X spreading in X-Z direction, and an extraction line 75Y indicating the extraction position of the tomographic image 80Y spreading in Y-Z direction are displayed. The medical image processing device 40 extracts and acquires the tomographic image 80X that passes through the extraction line 75X and spreads in Z direction (the depth direction) from the image area of the three-dimensional image 60. Further, the medical image processing device 40 extracts and acquires the tomographic image 80Y that passes through the extraction line 75Y and spreads in Z direction from the image area of the three-dimensional image 60.

In the present embodiment, the tomographic image 80X corresponds to a first tomographic image that extends in the depth direction (i.e., Z direction) and a first extending direction (X direction in the present embodiment), which is one of the directions that intersect the depth direction perpendicularly. Further, the tomographic image 80Y corresponds to a second tomographic image that extends in the depth direction (i.e., Z direction) perpendicularly and spreads in a second extending direction (i.e., Y direction) different from the first extending direction.

As described above, the three-dimensional image 60 in the present embodiment is formed by arranging, in Y direction, a plurality of tomographic images (B-scan images) 61 spreading in X-Z direction. Thus, the tomographic image 80X extending in X-Z direction may be at least one of the plurality of tomographic images 61 that form the three-dimensional image 60. Further, the tomographic image 80Y spreading in Y-Z direction spreads in Z direction (i.e., the depth direction) and a direction intersecting the B-scan image 61. In this case, the medical image processing device 40 extracts and acquires a tomographic image 80Y spreading in Y-Z direction from each of a plurality of B-scan images 61 included in the three-dimensional image 60.

The medical image processing device 40 may change the extraction position of the tomographic image 80 (80X, 80Y) from the three-dimensional image 60 according to an instruction input by a user. As one example, in the present embodiment, a user operates the operation unit 48 to change the extraction position of the tomographic image 80 by changing the position of at least one of the extraction line 75X and the extraction line 75Y appearing in the image area of the front image 70. The medical image processing device 40 changes the extraction position of the tomographic image 80 to a position on the changed extraction line 75X, 75Y in the image area of the three-dimensional image 60.

Details will be described later, but in the present embodiment, a first reference position serving as a reference for enlarging and minimizing the tomographic image 80X (the first tomographic image) in X direction (the first extending direction) may be an extraction position in the three-dimensional image at which the tomographic image 80Y (the second tomographic image) is extracted (in other words, the position of the extraction line 75Y). In this case, the extraction line 75Y appearing in the front image 70 indicates the first reference position. Further, a second reference position serving as a reference for enlarging and minimizing the tomographic image 80Y (the second tomographic image) in Y direction (the second extending direction) may be an extraction position in the three-dimensional image at which the tomographic image 80X (the first tomographic image) is extracted (in other words, the position of the extraction line 75X). That is, in the medical image processing device 40 in the present embodiment, a first reference line indicating the first reference position (the extraction line 75Y in the example shown in FIG. 5) and a second reference line indicating the second reference position (the extraction line 75X in the example shown in FIG. 5) can be displayed on the front image 70. However, in the medical image processing device 40, the first reference position and the second reference position may be set at positions deviated from the extraction lines 75X and 75Y. In such a case, the medical image processing device 40 may display the first reference position and the second reference position on the front image 75 separately from the extraction lines 75X and 75Y. In the present embodiment, the medical image processing device 40 also displays the first reference line indicating the first reference position on the tomographic image 80X (i.e., the first tomographic image). Further, the medical image processing device 41 also displays the second reference line indicating the second reference position on the tomographic image 80Y (i.e., the second tomographic image). The user may input an instruction for specifying the first reference position into the medical image processing device 40 by inputting an instruction for moving at least one of the first reference line on the front image 70 and the first reference line on the tomographic image 80X. Similarly, the user may input an instruction for specifying the second reference position into the medical image processing device 40 by inputting an instruction for moving at least one of the second reference line on the front image 70 and the second reference line on the tomographic image 80Y.

Based on the three-dimensional OCT data acquired by the imaging device 1, the medical image processing device 40 acquires (generates) an Enface image, which is a two-dimensional front image 70 when the tissue is viewed in a direction (a frontal direction) along the optical axis of the measurement light (an imaging light). The front image 70 shown in FIG. 5 is one example of an Enface image. Data of the Enface image may be, for example, integrated image data obtained by integrating luminance values in a depth direction (Z direction) at each position in X-Y direction, integrated values of spectral data at each position in X-Y direction, luminance data at each position in X-Y direction with a certain depth, and luminance data at each position in X-Y direction in any layer of a retina (for example, the retinal surface layer).

The medical image processing device 40 can generate motion contrast data (sometimes called as OCT angography data) by processing the OCT signals acquired by the imaging device 1. The motion contrast data is data obtained by processing a plurality of OCT signals acquired at different times at the same position on a living tissue. The motion contrast data includes information of the movement of a living tissue (for example, the movement of blood flow in a blood vessel in a living tissue, etc.).

The medical image processing device 40 acquires an angiographic front image by generating an Enface image based on motion contrast data. The front image 70 shown in FIG. 5 is one example of an angiography front image. By checking the angiography front image, a user can appropriately recognize the motion (for example, blood flow, blood vessels, etc.) of the living tissue from the front side of the tissue. In the present embodiment, an angiography frontal image (a blood vessel image) is generated that is an image indicating the position of the blood vessel in a specific layer by generating an Enface image of the specific layer in the fundus tissue based on the motion contrast data.

As shown in FIG. 5, the medical image processing device 40 in the present embodiment superimposes motion contrast data information on the tomographic images 80 (80X, 80Y). As described above, the motion contrast data includes information of movement in a tissue (for example, a movement of blood flow in a blood vessel in a living tissue, etc.). Therefore, the user can appropriately recognize the movement (for example, blood flow, blood vessels, etc.) in the living tissue on the tomographic images 80 by checking the tomographic images 80 on which the motion contrast data information is superimposed.

### First Embodiment

With reference to FIGS. 6 to 8, an image display process executed by the medical image processing device 40 in the first embodiment will be described. In the present embodiment, the medical image processing device 40 which is an OCT acquires data of an image of a living tissue from the imaging device 1, and executes a display process on the acquired image. However, as described above, other devices may serve as the medical imaging device. For example, the imaging device (the OCT device in the present embodiment) 1 itself may execute the image display process. Further, a plurality of control units (for example, the CPU 31 of the imaging device 1 and the CPU 41 of the medical image processing device 40) may collaboratively execute the image display process. In the present embodiment, the CPU 41 of the medical image processing device 40 executes the image display process shown in FIG. 6 according to the medical image processing program stored in the NVM 44.

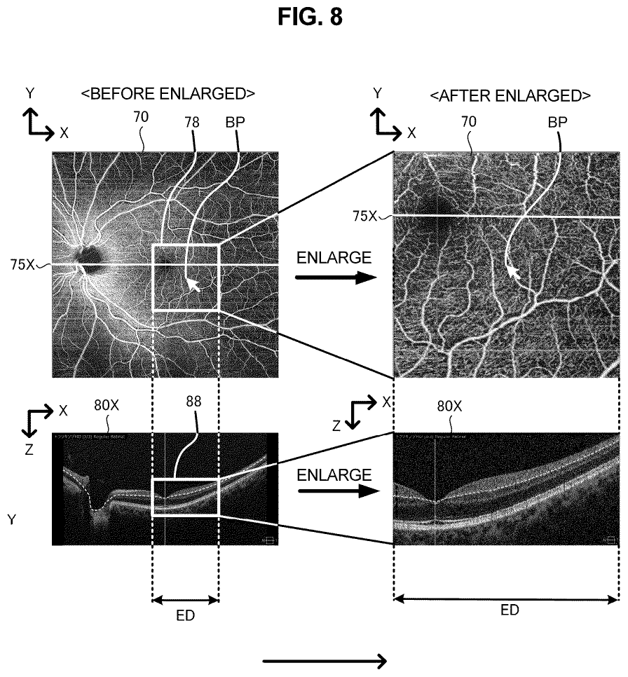

First, the CPU 41 acquires data of a three-dimensional image 60 (see FIG. 4) of the living tissue captured by the imaging device 1 (S1). The CPU 41 acquires a front image 70 (see FIGS. 5 and 8) from the three-dimensional image 60 acquired at S1 (S2). As described above, in the present embodiment, an Enface image is generated based on the three-dimensional OCT data, and then the front image 70 is acquired. More specifically, in the present embodiment, an angiographic front image is acquired by generating an Enface image based on the motion contrast data.

However, the CPU 41 may acquire a front image at S2 that is different from the angiography front image. For example, the CPU 41 may acquire a front image by generating an Enface image based on three-dimensional OCT data different from the motion contrast data. The CPU 41 may acquire a thickness map showing a two-dimensional distribution of the thickness of a particular layer in the living tissue as a front image. Further, by inputting a three-dimensional image into a mathematical model trained by a machine learning algorithm, the CPU 41 may acquire a probability distribution for identifying a specific structure (for example, at least one of a layer and a boundary) in the living tissue appearing in the three-dimensional image. The CPU 41 may acquire, as a front image, a deviation degree map showing a two-dimensional distribution of the degree of deviation of the acquired probability distribution with respect to the probability distribution that will be generated when the target structure is accurately identified. The CPU 41 may acquire front image data taken by a device different from the OCT device (for example, at least one of a fundus camera, a scanning laser ophthalmoscope (SLO), an infrared camera, and the like).

Return to the description of FIG. 6. The CPU 41 sets the extraction position for the tomographic image 80 to the default position in the three-dimensional image 60 acquired at S1 (S3). The CPU 41 acquires a tomographic image 80 (see FIGS. 5 and 8) by extracting a two-dimensional image at the extraction position set at S3 from the three-dimensional image 60 (S4). As shown in FIG. 5, in the present embodiment, the extraction lines 75X and 75Y are set on the front image 70, so that the extraction positions for the tomographic images 80X and 80Y are set. The CPU 41 extracts, from the three-dimensional image 60, the tomographic images 80X and 80Y that pass through the corresponding extraction line 75X or 75Y and extend in the depth direction (i.e., Z direction). Note that the default position to which the extraction position is set at S3 can be set appropriately. As an example, in the present embodiment, a default extraction line 75X is set at the center position in Y direction in the image range of the three-dimensional image 60. Further, a default extraction line 75Y is set at the center position in X direction in the image range of the three-dimensional image 60.

However, the tomographic image acquired at S4 is not necessarily limited to the tomographic image taken by the OCT device. A tomographic image taken by an MRI (magnetic resonance imaging) device, a CT (computed tomography) device, or the like may be acquired.

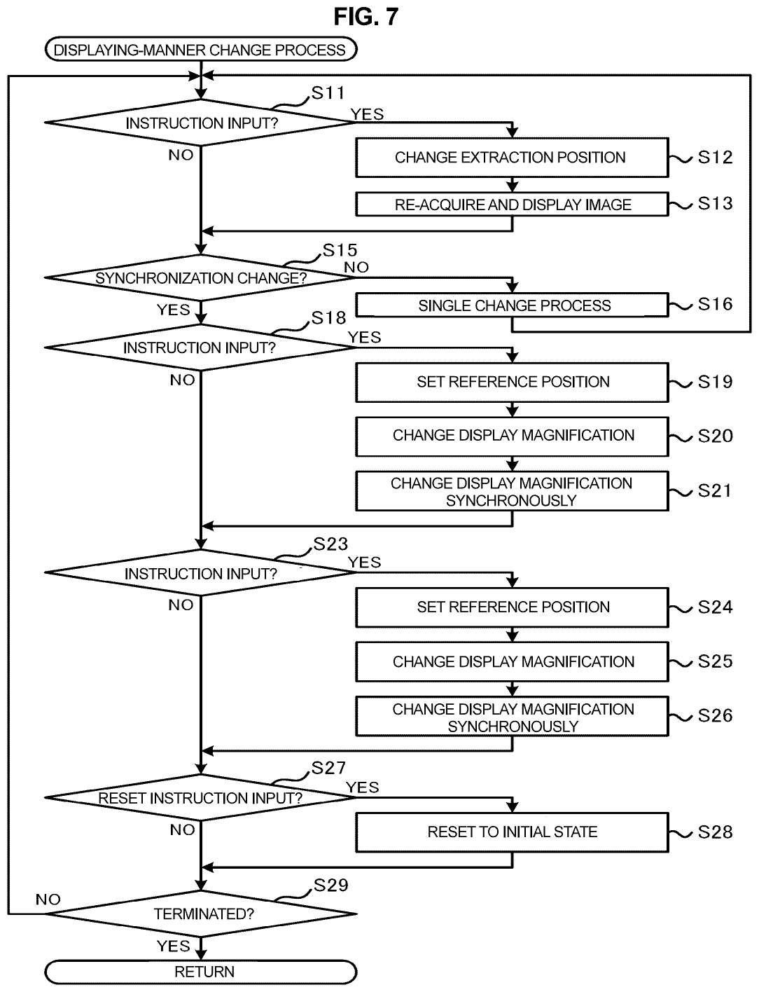

The CPU 41 displays the front image 70 acquired at S2 and the tomographic images 80 acquired at S4 on the monitor 47 (S5). Next, a displaying-manner change process (S6, see FIG. 7) is executed.

As shown in FIG. 7, when the displaying-manner change process is started, the CPU 41 determines whether an instruction (trigger) to change the extraction position for the tomographic image 80 from the three-dimensional image 60 has been input (S11). If not input (S11: NO), the process proceeds to S15. As described above, in the present embodiment, an instruction (a trigger) may be input by a user operating the operation unit 48 to change the extraction position of the tomographic image 80 by changing the position of at least one of the extraction line 75X and the extraction line 75Y appearing in the image area of the front image 70. Further, the trigger to change the extraction position may be entered automatically. For example, the CPU 41 may automatically output a trigger to change the extraction position to a position in accordance with the image analysis result. In this case, a specific method for changing the extraction position according to the image analysis result may be appropriately selected. For example, the extraction position for the tomographic image may be automatically changed to a position where it is determined from the angography front image that blood flow is likely to deteriorate or to a position where the degree of deviation indicated by the deviation map is equal to or greater than a threshold value. When the instruction to change the extraction position is input (S11: YES), the CPU 41 changes the extraction position for the tomographic image 80 to a position that passes the changed extraction lines 75X, 75Y in the image area of the three-dimensional image 60 (S12). The CPU 41 acquires the tomographic image 80 again from the new extraction position in the three-dimensional image 60 and displays the acquired image 80 on the monitor 47 (S13). After that, the process proceeds to S15.

The CPU 41 determines whether an instruction to execute a magnification synchronization change process has been input by the user (S15). The medical image processing device 40 in the present embodiment executes a magnification synchronization change process (S18-S29) and a magnification single change process (S16). In the magnification synchronization change process, both a display magnification for the front image 70 and a display magnification for the tomographic image 80 are changed synchronously by the user simply inputting a display magnification change instruction for either the front image 70 or the tomographic image 80 that are displayed on the monitor 47. On the other hand, in the magnification single change process, the display magnification only for the front image 70 or the tomographic image 80 to which the display magnification change instruction was given is changed solely among the front image 70 and the tomographic image 80 displayed on the monitor 47. By operating the operation unit 48, the user can input an instruction for specifying whether to execute the magnification synchronization change process or the magnification single change process. When the instruction for executing the magnification synchronization change process is input (S15: YES), the CPU 41 executes the magnification synchronization change process (S18-S29) described below. On the other hand, when the instruction for executing the magnification single change process is input (S15: NO), the CPU 41 executes the magnification single change process (S16) according to the instruction input by the user. The steps of S11-S16 are repeated. As described above, the user can select the magnification synchronization change process or the magnification single change process according to various situations.

Hereinafter, the magnification synchronization change process (S18-S29) will be described in detail. In the present embodiment, all display magnifications for the front image 70, the tomographic image 80X extending in X-Z direction, and the tomographic image 80Y extending in Y-Z direction are changed synchronously. However, in the following (FIG. 8, etc.), the display magnification of the front image 70 and the display magnification of the tomographic image 80X extending in X-Z direction may be synchronously changed in order to simplify the description. It should be noted that, when synchronously changing the display magnification of the front image 70 and the display magnification of the tomographic image 80Y extending in Y-Z direction, the method described below may be applied with replacing “X direction” with “Y direction”.

The CPU 41 determines whether the display magnification change instruction for the front image 70 displayed on the monitor 47 has been input to the monitor 47 (S18). If not input (S18: NO), the process proceeds to S23. A method for causing the user to input the display magnification change instruction for the front image 70 may be appropriately selected. As one example, in the present embodiment, the CPU 41 displays, on the monitor 47, a cursor that moves in the display area according to the operation of the operation unit 48 (a mouse in the present embodiment). The user operates the operation unit 48 to move the cursor displayed on the monitor 47 to a position (hereinafter, referred to as a “reference position”) that is the center of the change (enlarging or minimizing) of the display magnification of the front image 70. Thereafter, the user performs an operation to enlarge or minimize the image (in the present embodiment, an operation of forward or reverse rotation of the mouse wheel). As a result, an instruction for changing the display magnification for the front image 70 and an instruction for specifying the reference position in the front image 70 are input into the medical image processing device 40. Therefore, the user can enlarge or minimize the image based on the specified reference position by specifying an appropriate position as the reference position.

When the instruction for changing the display magnification for the front image 70 is input (S18: YES), the CPU 41 sets the reference position for changing the magnification of the front image 70 at a position in the front image 70 specified by the user (in the present embodiment, the position of the cursor on the front image 70 at which the rotation operation of the mouse wheel was performed) (S19). The CPU 41 executes a change process (an enlarging process or a minimizing process) of the display magnification of the front image 70 based on the reference position set at S19 (S20).

In FIG. 8, the front image 70 and the tomographic image 80X before enlarged are shown on the left side, and the front image 70 and the tomographic image 80X after enlarged are shown on the right side. In the example shown in FIG. 8, an instruction to enlarge the front image 70 is input with the cursor at a slightly lower right position from the center of the front image 70. Therefore, the CPU 41 sets the reference position at the position of the cursor on the front image 70 and enlarges the front image 70 based on the reference position in a display frame for the front image 70 on the monitor 47. As a result, the area in the rectangular frame 78 centered on the cursor position in the front image 70 before enlarged (i.e., the left side of FIG. 8) is enlarged and displayed in the display frame of the monitor 47.

Further, the CPU 41 executes a process (S21) for changing the display magnification of the tomographic images 80X, 80Y together with the process (S19 and S20) for changing the display magnification of the front image 70. At the S21 in the present embodiment, the CPU 41 changes the display magnification of the tomographic image 80X while synchronizing, the display range of the tomographic image 80X on the monitor 47 in the extending direction (i.e., X direction in FIG. 8) perpendicular to the depth direction (i.e., Z direction) and the display range of the front image 80X on the monitor 47 in the extending direction (i.e., X direction in FIG. 8). In the example shown in FIG. 8, both the display magnification of the front image 70 and the display magnification of the tomographic image 80X are changed while synchronizing the display image of the tomographic image 80X in X direction and the display range of the front image 70 in X direction (i.e., the display ranges commonly indicated by “ED” in FIG. 8). As a result, even if the display magnification of both the front image 70 and the tomographic image 80X is changed synchronously, the display range of the front image 70 in the extending direction matches the display range of the tomographic image 80X in the extending direction. Thus, the user can easily compare the front image 70 with the tomographic image 80X on the site of interest.

In addition, when changing the display magnification of the tomographic image 80 at S21, a method for determining the display position for the tomographic image 80X in the depth direction (Z direction) may be appropriately selected. For example, the CPU 41 may perform a layer/boundary acquisition process for acquiring the position of a specific layer or boundary (hereinafter, simply referred to as a “layer/boundary”) among the living tissue appearing in the tomographic image 80X displayed on the monitor 47. At S21, the CPU 41 may change the display magnification of the tomographic image 80X while maintaining an average position in the depth direction (i.e., Z direction) of the specific layer/boundary in the tomographic image 80X to a target position in the depth direction (for example, a predetermined position or a position calculated according to the display magnification). In this case, the specific layer/boundary can properly appear (be shown) around the predetermined position in the depth direction regardless of the display magnification of the tomographic image 80X. Therefore, even when the display magnification of the front image 70 and the tomographic image 80X is changed, the user can appropriately observe the specific layer/boundary in the tomographic image 80X. In addition, a mathematical model trained by a machine learning algorithm may be used at the layer/boundary acquisition process. The position of the layer/boundary determined by an operator may be acquired as it is.

When the display magnification of the tomographic image 80 is changed, the target position for maintaining the position of the specific layer/boundary in the depth direction can be appropriately selected. For example, the CPU 41 may change the display magnification of the tomographic image while maintaining the average position of the specific layer/boundary in the depth direction at the center of the depth direction in the display range in the image. Alternatively, the CPU 41 may change the display magnification of the tomographic image while maintaining the average position of the specific layer/boundary in the depth direction at the position of the specific layer/boundary immediately before changing the display magnification. Yet alternatively, the CPU 41 may specify a range (hereinafter, referred to as a “slab”) of the layer/boundary in the depth direction for which the Enface image described above is generated. Then, the CPU 41 may change the display magnification of the tomographic image by setting a predetermined position (for example, the center position) in the specified slab in the depth direction as the center for changing the display magnification. The slab can be identified by the boundary on the surface side and the boundary on the deep side of the range within which the Enface image is generated. The boundary for specifying the slab may be a layer in the living body that actually appears in the image or may be a position that is offset from the layer of the living body a predetermined distance in the depth direction. By changing the display magnification of the tomographic image 80 based on the slab, the specific layer/boundary can be observed more appropriately.

Further, at S21, the CPU 41 may change the display magnification of the tomographic image 80X only in the extending direction (X direction in FIG. 8) intersecting the depth direction synchronously with the display magnification of the front image 70 without changing the display magnification of the tomographic image 80X in the depth direction (i.e., Z direction). In this case, even if the tomographic image 80X is enlarged, at least one of the layer and the boundary (hereinafter, simply referred to as a “layer/boundary”) in which the user is interested is less likely to be displayed at a position outside of the display range of the tomographic image 80X. Therefore, the user can more appropriately observe the layer/boundary of interest on the tomographic image 80X. Further, the CPU 41 may change the display magnification of the tomographic image 80X so that the center position of the tomographic image 80X in the depth direction is always aligned with the center position of the display frame of the tomographic image 80X in the depth direction. Further, at S21, the CPU 41 may change the display magnification of both the images while maintaining both the aspect ratio of the front image 70 and the aspect ratio of the tomographic image 80X.

Further, the specific method for synchronously changing the display magnification of the front image 70 and the tomographic image 80X can also be appropriately selected. For example, the CPU 41 may synchronously change the display magnification of the front image 70 and the display magnification of the tomographic image 80X based on imaging information of each of the front image 70 and the tomographic image 80X (for example, the imaging angle of the front image 70 and the tomographic image 80X, the number of scanning points, resolution, the model of the imaging device 1, and the axial length of the subject eye when the living tissue is an eye).

In the present embodiment, when the instruction for changing the display magnification of the front image 70 is input, not only the display magnification of the tomographic image 80X extending in X-Z direction but also the display magnification of the tomographic image 80Y extending in Y-Z direction is changed synchronously. To synchronously change the display magnification of the front image 70 and the display magnification of the tomographic image 80Y extending in Y-Z direction, the method at S21 may be applied with replacing “X direction” with “Y direction”. Further, the CPU 41 may synchronously change the display magnification of the tomographic image 80X and the display magnification of the tomographic image 80Y while matching the display range of the tomographic image 80X in the depth direction and the display range of the tomographic image 80Y in the depth direction. In this case, since the display ranges of the plurality of tomographic images 80X, 80Y in the depth direction match, the condition or the status of the tissue can be more appropriately recognized.

Next, the CPU 41 determines whether a display magnification change instruction for the tomographic image 80 displayed on the monitor 47 (in the present embodiment, either the tomographic image 80X or the tomographic image 80Y) has been input into the monitor 47 (S23). If not input (S23: NO), the process proceeds to S27. A method for causing the user to input the display magnification change instruction for the tomographic image 80 may be appropriately selected. As one example, in the present embodiment, the CPU 41 displays, on the monitor 47, a cursor that moves in the display area according to the operation of the operation unit 48 (a mouse in the present embodiment). The user operates the operation unit 48 to move the cursor displayed on the monitor 47 to a position (hereinafter, referred to as a “reference position”) that serves as the center of the change (enlarging or minimizing) of the display magnification for the tomographic image 80. Thereafter, the user performs an operation to enlarge or minimize the image (in the present embodiment, an operation of forward or reverse rotation of the mouse wheel). As a result, an instruction for changing the display magnification for the tomographic image 80 and an instruction for specifying the reference position in the tomographic image 80 are input into the medical image processing device 40. Therefore, the user can enlarge or minimize the image based on the specified reference position by specifying an appropriate position as the reference position.

When the instruction for changing the display magnification for the tomographic image 80 is input (S23: YES), the CPU 41 sets the reference position for changing the magnification of the tomographic image 80 at a position in the tomographic image 80 specified by the user (in the present embodiment, the position of the cursor on the tomographic image 80 at which the rotation operation of the mouse wheel was performed) at S24. The CPU 41 executes a process (an enlarging process or a minimizing process) for changing the display magnification of the front tomographic 80 based on the reference position set at S24 as the center (S25).

Further, the CPU 41 executes a process (S26) for changing the display magnification of the front image 70 in addition to the process (S24 and S25) for changing the display magnification of the tomographic image 80. At the S26 of the present embodiment, the CPU 41 changes the display magnification of the front image 70 while synchronizing the display range of the tomographic image 80X or the tomographic image 80Y on the monitor 47 in the extending direction perpendicular to the depth direction (i.e., Z direction) and the display range of the front image 70 on the monitor 47 in the extending direction with each other. Here, if the instruction for changing the display magnification is given to the tomographic image 80X, the extending direction is X direction. If the instruction is given to change the display magnification of the tomographic image 80Y, the extending direction is Y direction. Note that the tomographic image 80 may extend in a direction intersecting each of X direction and Y direction. The display range of the front image 70 on the monitor 47 in the extending direction is synchronized with the display range of the tomographic image 80 in the extending direction. By performing the step of S26, the user can more appropriately compare the front image 70 with the tomographic image 80X of the site of interest.

It should be noted that, when the display magnification of the front image 70 is changed at S26, a method for determining the display position of the front image 70 in an intersecting direction that intersects the extending direction (for example, when the extending direction is X direction, the intersecting direction is Y direction) can be appropriately selected. For example, the CPU 41 may change the display magnification of the front image 70 while maintaining the center position of the front image 70 in the intersecting direction. Further, the CPU 41 may change the display magnification of the front image 70 while maintaining, at a fixed position regardless of the display magnification, the position of the extraction line 75 (75X or 75Y) extending in the extending direction in the display area of the front image 70. In this case, since the relative positional relationship between the acquired front image 70 and the tomographic image 80 is appropriately maintained, the condition of the living tissue can be appropriately acquired.

When an instruction for changing the display magnification is input for one of the plurality of tomographic images 80X and 80Y, the CPU 41 synchronously changes the display magnification of the other of tomographic images 80X and 80Y. As one example, the CPU 41 changes the display magnification of the plural tomographic images 80 while synchronizing the display range of the tomographic image 80 in the depth direction for which the display magnification change instruction is given and the display range of the other tomographic image 80 in the depth direction with each other. Further, the CPU 41 changes the display magnification of the other tomographic image 80 while synchronizing the display range of the other tomographic image 80 in the extending direction (i.e., a direction intersecting the depth direction) with the display range of the front image 70 in the extending direction.

Further, the CPU 41 may set the first reference position in the tomographic image 80X (the first tomographic image) in X direction (the first extending direction) that serves as a reference for expanding and minimizing (i.e., the center of expansion/minimization in the present embodiment). The CPU 41 may set the second reference position in the tomographic image 80Y (the second tomographic image) in Y direction (the second extending direction) that serves as a reference for expanding and minimizing (i.e., the center of expansion/minimization in the present embodiment). The CPU 41 may synchronously change the display magnification of the tomographic image 80X and the display magnification of the tomographic image 80Y by setting the first reference position in the tomographic image 80X in the X direction as the center of expansion and minimization and the second reference position in the tomographic image 80Y in Y direction as the center of expansion and minimization. The CPU 41 may synchronously change the display magnification of the front image, the display magnification of the tomographic image 80X, and the display magnification of the tomographic image 80Y by setting the center of expansion and minimization in the X direction for the front image 70 as the first reference position, and the center of expansion and minimization in Y direction for the second reference position. In this case, the display magnification of the plural images is synchronously changed based on the set first reference position and the second reference position.

A specific method for setting the first reference position and the second reference position can be appropriately selected. For example, as described above, the CPU 41 may automatically set the first reference position at the position in X direction in the three-dimensional image 60 where the tomographic image 80Y is extracted (in the present embodiment, the position of the extraction line 75Y). The CPU 41 may automatically set the second reference position at the position in the three-dimensional image 60 in Y direction where the tomographic image 80X is extracted (in the present embodiment, the position of the extraction line 75X). In this case, the display magnification of one of the tomographic images is changed based on the position at which the other of the tomographic images is extracted. Therefore, the user can easily compare the plurality of images.

Further, the CPU 41 may set at least one of the first reference position and the second reference position in response to an instruction input by a user. In this case, the user can change the display magnification of at least one of the first tomographic image and the second tomographic image based on the desired position. More specifically, the user may input an instruction for specifying the first reference position into the medical image processing device 40 by inputting an instruction for moving at least one of the first reference line on the front image 70 and the first reference line on the tomographic image 80X. Similarly, the user may input an instruction for specifying the second reference position into the medical image processing device 40 by inputting an instruction for moving at least one of the second reference line on the front image 70 and the second reference line on the tomographic image 80Y.

Next, the CPU 41 determines whether a reset instruction has been input to return back the display magnification and the display position of the image to an initial state for the image (S27). If not input (S27: NO), the process proceeds to S29. When the reset instruction is input (S27: YES), the CPU 41 resets at least one of the display magnification of the front image 70 and the tomographic image 80 and the display position in the display frame of the monitor 47 to the initial state at the time of starting the display (S28). Therefore, the user can return the display magnification and the display position of the front image 70 and the tomographic image 80 back to the state at the time of starting the display with a simple operation.

If an instruction to terminate the process is not input (S29: NO), the process returns back to S11, and the steps of S11-S29 are repeated. When the termination instruction is input (S29: YES), the image display process is terminated.

According to the present embodiment, both the display magnification for the front image 70 and the display magnification for the tomographic image 80 are changed synchronously by inputting, into the medical image processing device 40, the display magnification change instruction for either the front image 70 or the tomographic image 80 that are displayed on the monitor 47. Thus, the user can easily and appropriately compare the front image 70 and the tomographic image 80 as compared to individually changing the magnification of the front image 70 and the magnification of the tomographic image 80.

Further, in the present embodiment, both the angiography front image and the tomographic image on which the motion contrast data information is superimposed (hereinafter, referred to as “MC superimposed tomographic image”) are displayed on the monitor 47. Therefore, the user can appropriately recognize the movement such as blood flow in a living tissue from both the angiography front image and the MC superimposed tomographic image. For example, the state of the tissue for the portion that appears as a blood vessel in the front image 70 can be checked on the tomographic image 80. Further, according to the present embodiment, the display magnification of both the angiography front image and the MC superimposed tomographic image is changed synchronously by inputting an instruction to change the display magnification for only one of the images. Therefore, by comparing the angiography front image with the MC superimposed tomographic image in which the display magnifications are changed synchronously, the user can more easily and appropriately recognize the movement such as blood flow in a living tissue.

### Second Embodiment

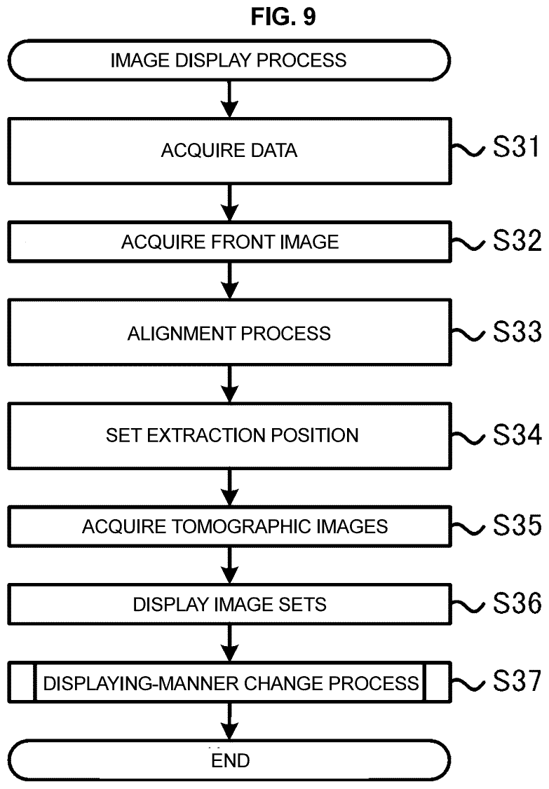

With reference to FIGS. 9 to 11, an image display process executed by a medical image processing device 40 in a second embodiment will be described. It should be noted, for some of the processes in the second embodiment, the same processing as described in the first embodiment described above can be applied. Therefore, in the following, when the same process as in the first embodiment can be used, the description is omitted or simplified. The CPU 41 of the medical image processing device 40 executes the image display process shown in FIG. 9 according to a medical image processing program stored in the NVM 44.

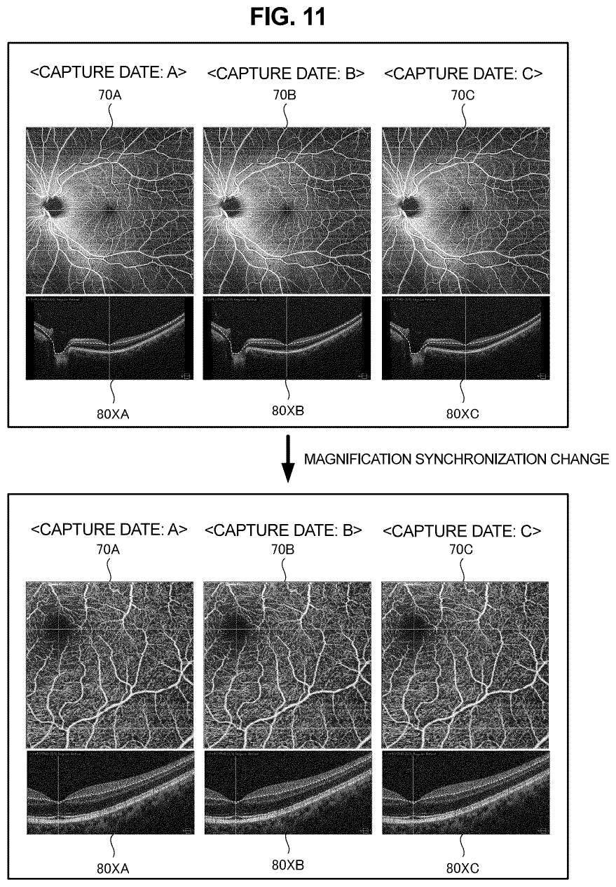

In the second embodiment, all display magnifications of the front image 70, the tomographic image 80X extending in X-Z direction, and the tomographic image 80Y extending in Y-Z direction are changed synchronously. However, in the following (FIG. 11, etc.), the display magnification of the front image 70 and the display magnification of the tomographic image 80X extending in X-Z direction may be synchronously changed in order to simplify the description as with the first embodiment. It should be noted that, when synchronously changing the display magnification of the front image 70 and the display magnification of the tomographic image 80Y extending in Y-Z direction, the method described below may be applied with replacing “X direction” with “Y direction”.

First, the CPU 41 acquires data from the plurality of three-dimensional images 60 (see FIG. 4) taken from the same living tissue of the same subject (S31). Here, the timings for imaging the plurality of three-dimensional images 60 acquired at S31 may be different from each other. In this case, by executing the process described below, the user can more easily and appropriately observe (that is, follow-up) changes in the subject living tissue over time.

The CPU 41 acquires the front image 70 from each of the plurality of three-dimensional images 60 acquired at S31. In the example shown in FIG. 11, a first front image 70A is acquired from the first three-dimensional image 60A, a second front image 70B is acquired from the second three-dimensional image 60B, and a third front image 70C is acquired from the third three-dimensional image 60C. In the second embodiment, as with the first embodiment, the plurality of front images 70 are acquired by generating an Enface image based on each of the plurality of three-dimensional OCT data. More specifically, a plurality of angiographic front images are acquired by generating an Enface image based on each of a plurality of pieces of the motion contrast data. However, as described above, a front image different from the angiography front image may be acquired.

Next, the CPU 41 executes an alignment process for the plurality of three-dimensional images 60 acquired at S31 (S33). As one example, in the present embodiment, the CPU 41 performs the alignment process (a registration process) for the plurality of front images 70 acquired at S32 to align the positions of the plurality of three-dimensional images 60 when viewed in a front direction. As a result, the user can appropriately compare living tissues that appear in the plurality of front images 70 at the same position. Further, in the present embodiment, alignment of the plurality of three-dimensional images 60 is also performed using the front image 70. Therefore, the positions of the living tissues appearing in the plurality of tomographic images 80 acquired at S35 described later can easily match. The alignment process includes at least one of rotation, translation, and scaling processes (all the processes in the present embodiment).

The CPU 41 sets the extraction position of the tomographic image 80 to the same position (i.e., the default position) in each of the plurality of three-dimensional images 60 acquired at S31 (S34). The CPU 41 acquires a plurality of tomographic images 80 (see FIG. 11) by extracting a two-dimensional image at the extraction position set at S34 from each of the plurality of three-dimensional images 60 (S35). As a result, the imaging positions of the extracted tomographic images 80 in the living tissue are common to each other. Further, as described above, in the present embodiment, the tomographic images 80 are extracted from the plurality of three-dimensional images 60 at the same position after aligning the plurality of three-dimensional images 60 when viewed in at least the front direction. Therefore, the imaging positions of the plurality of tomographic images 80 in the living tissue can be close to each other. In the example shown in FIG. 11, a first tomographic image 80XA is extracted from a first three-dimensional image 60A, a second tomographic image 80XB is extracted from a second three-dimensional image 60B, and a third tomographic image 80XC is extracted from a third three-dimensional image 60C. The imaging positions of the first tomographic image 80XA, the second tomographic image 80XB, and the third tomographic image 80XC in each living tissue are close to each other. Although the illustration is omitted, in the present embodiment, the tomographic images 80 extending in Y-Z direction are also extracted from the three three-dimensional images 60A˜60C at the same position. As with the first embodiment, a default position at which the extraction position is set can be set appropriately.

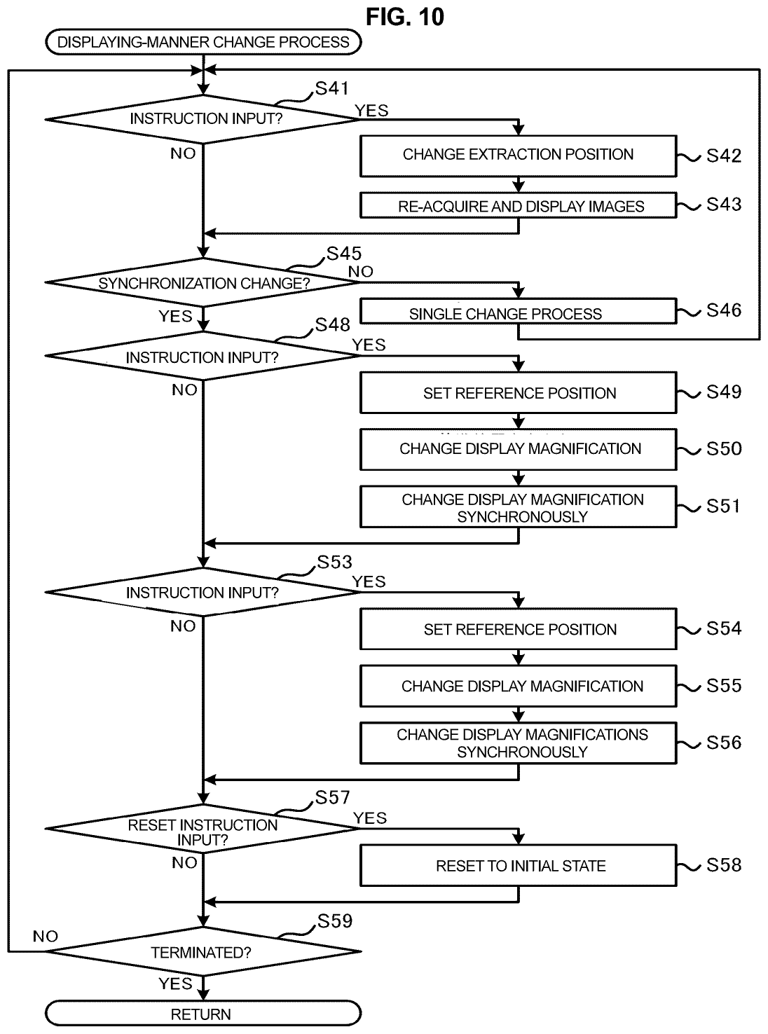

The CPU 41 displays a plurality of image sets (a set of the front image 70 and the tomographic image 80) acquired from the plurality of three-dimensional images 60 on the monitor 47 (S36). In the example shown in FIG. 11, a first image set (the first front image 70A and the first tomographic image 80XA) acquired from the first three-dimensional image 60A, a second image set (the second front image 70B and the second tomographic image 80XB) acquired from the second three-dimensional image 60B, and a third image set (the third front image 70C and the third tomographic image 80XC) acquired from the third three-dimensional image 60C are displayed on the monitor 47. Next, the displaying-manner change process (S37, see FIG. 10) is executed.

As shown in FIG. 10, when the displaying-manner change process is started, the CPU 41 determines whether an instruction (trigger) to change the extraction positions for the tomographic images 80 from the three-dimensional images 60A to 60C has been input (S41). As described above, the instruction (a trigger) for changing the extraction positions for the tomographic images 80 may be input by the user or may be input automatically. If not input (S41: NO), the process proceeds to S45. In the second embodiment, the user operates the operation unit 48 to input an instruction for changing the extraction position of one of the plurality of tomographic images 80 from the three-dimensional image 60 by changing the position of the extraction line 75X, 75Y in one of the plurality of front images 70 displayed on the monitor 47. When the instruction for changing the extraction position of one of the tomographic images 80 is input, the CPU 41 changes, to the same position, the extraction positions for all the displayed tomographic images 80 from the three-dimensional image 60 (S42). The CPU 41 re-acquires the tomographic images 80 from the plurality of three-dimensional images 60 at the new extraction position and displays the images 80 on the monitor 47 (S43). At the process of S33 described above, the alignment process on the plurality of three-dimensional images 60 has been performed in advance. Therefore, when the step of S43 is performed, the user can change the extraction positions for the plurality of tomographic images 80 to the same position at once by simply changing the extraction position of one of the plurality of tomographic images 80.

The CPU 41 determines whether an instruction to execute a magnification synchronization change process has been input by the user (S45). The medical image processing device 40 in the second embodiment executes the magnification synchronization change process (S48-S59) and a magnification single change process (S46). At the magnification synchronization change process, the display magnification for all the plurality of front images 70 and the plurality of tomographic images 80 that are displayed are changed synchronously by the user simply inputting the display magnification change instruction for one of the front and tomographic images 70, 80 among the plurality of the front images 70 and the plurality of tomographic images 80 that are displayed on the monitor 47. On the other hand, in the magnification single change process, the display magnification only for one of the plurality of front and tomographic images 70, 80 to which the display magnification change instruction is given is changed solely among the front images 70 and the tomographic images 80 displayed on the monitor 47. By operating the operation unit 48, the user can input an instruction for specifying whether to execute the magnification synchronization change process or the magnification single change process. When the instruction for executing the magnification synchronization change process is input (S45: YES), the CPU 41 executes the magnification synchronization change process (S48-S59) described below. On the other hand, when the instruction for executing the magnification single change process is input (S45: NO), the CPU 41 executes the magnification single change process (S46) according to the instruction input by the user. The steps of S41-S46 are repeated. As described above, the user can select the magnification synchronization change process or the magnification single change process according to various situations.

Hereinafter, the magnification synchronization change process (S48-S59) will be described in detail. The CPU 41 determines whether the display magnification change instruction has been input into the monitor 47 for one of the plurality of front images 70 displayed on the monitor 47 (S48). If not input (S48: NO), the process proceeds to S53. As one example in the second embodiment, the user operates the operation unit 48 to move the cursor displayed on the monitor 47 to a position (hereinafter, referred to as a “reference position”) that serves as the center of the change (enlarging or minimizing) of the display magnification of one of the plurality of front images 70. Thereafter, the user performs an operation to enlarge or minimize the image (in the present embodiment, an operation of forward or reverse rotation of the mouse wheel). As a result, an instruction to change the display magnification for one of the front images 70 and an instruction for specifying the reference position for the front image 70 are input into the medical image processing device 40.

When the instruction for changing the display magnification for one of the plurality of front images 70 is input (S48: YES), the CPU 41 sets the reference position at a position in the front image 70 specified by the user (in the present embodiment, the position of the cursor on the front image 70 at which the rotation operation of the mouse wheel was performed). Further, the CPU 41 also sets, at the same position in, the reference positions for other front images 70 among the plurality of front images 70 for which the display magnification change instruction is not input (S49). The CPU 41 synchronously executes a process (an enlarging process or a minimizing process) for changing the display magnification of each of the plurality of front images 70 with the reference position set at S49 as the center (S50). That is, the CPU 41 sets the reference position for enlargement and minimization at the same position in the image area of each of the plurality of front images 70, and executes an enlarging process or a minimizing process on the plurality of front images 70 based on the set reference positions. As a result, the display magnifications of the plurality of front images 70 are synchronously changed while aligning the display positions of the plurality of front images 70.

In FIG. 11, the plurality of front images 70 and the plurality of tomographic images 80X before enlarged are shown on the upper side, and the plurality of front images 70 and the plurality of tomographic images 80X after enlarged are shown on the lower side. In the example shown in FIG. 11, each of the plurality of front images 70 is enlarged based on the same reference position in each image area.

Further, the CPU 41 executes a process (S51) for synchronously changing the display magnifications of the tomographic images 80X, 80Y in addition to the process (S49 and S50) for changing the display magnifications of the plurality of front images 70. The method for changing the display magnification of each tomographic image 80 can be the same as the method described at S21 in the first embodiment. Therefore, this detailed description is omitted.

Next, the CPU 41 determines whether a display magnification change instruction for the plurality of tomographic images 80 displayed on the monitor 47 (in the present embodiment, one of the plurality of tomographic images 80X and the plurality of tomographic images 80Y) has been input into the monitor 47 (S53). If not input (S53: NO), the process proceeds to S57. As one example in the present embodiment, the user operates the operation unit 48 to move the cursor displayed on the monitor 47 to a position (hereinafter, referred to as a “reference position”) that serves as the center for the change (enlarging or minimizing) of the display magnification of one of the plurality of tomographic images 80. Thereafter, the user performs an operation to enlarge or minimize the image (in the present embodiment, an operation of forward or reverse rotation of the mouse wheel). As a result, an instruction to change the display magnification for one of the tomographic images 80 and an instruction for specifying the reference position in the tomographic image 80 are input into the medical image processing device 40.

When the instruction for changing the display magnification for one of the tomographic images 80 is input (S53: YES), the CPU 41 sets the reference position at a position in the tomographic image 80 (the tomographic image 80X or the tomographic image 80Y) specified by the user (in the present embodiment, the position of the cursor on the tomographic image 80 at which the rotation operation of the mouse wheel was performed). Further, the CPU 41 sets, at the same position, the reference positions for the tomographic images 80X or the tomographic images 80Y for which the instruction for changing the display magnification is not input among the plurality of tomographic images 80X or the plurality of tomographic images 80Y that extend in the first extending direction (i.e., X direction or Y direction) that is the same direction as the extending direction of the tomographic image 80 for which the instruction for changing the display magnification is input. The CPU 41 synchronously changes the display magnifications of the plurality of tomographic images 80 (the tomographic images 80X or the tomographic images 80Y) extending in the first extending direction with the reference position set at S54 as the center (S55). That is, the CPU 41 sets the reference position for enlargement and minimization at the same position in the image area of each of the plurality of tomographic images 80 extending the first extending direction, and executes an enlarging process or a minimizing process on the plurality of tomographic images 80 extending in the first extending direction based on the set reference position. As a result, the display magnifications of the plurality of tomographic images 80 are synchronously changed while aligning the display positions of the plurality of tomographic images 80.

Further, the CPU 41 executes a process (S56) for changing the display magnifications of the plurality of front images 70 as well as the process (S54, S55) for changing the display magnifications of the plurality of tomographic images 80 extending in the first extending direction. At the S56 in the present embodiment, the CPU 41 changes the display magnifications of the plurality of front images 70 while synchronizing the display range of the tomographic image 80 on the monitor 47 in the first extending direction (a direction perpendicular to the depth direction) and the display range of the front image 70 on the monitor 47 in the first extending direction with each other. At S56, when the display magnification of the front image 70 is changed, a method for determining the display position of the front image in the second extending direction intersecting the first extending direction can be appropriately selected. For example, the CPU 41 may change the display magnifications of the plurality of front images 70 while maintaining the center position of each front image 70 in the second extending direction. Further, the CPU 41 may change the display magnifications of the plurality of front images 70 while maintaining, at a fixed position regardless of the display magnification, the position of the extraction line 75 (75X or 75Y) extending in the first extending direction in the display area of each of the plurality of front images 70. In this case, since the relative positional relationship between the acquired front image 70 and the tomographic image 80 is appropriately maintained, the condition of the living tissue can be appropriately acquired. In the present embodiment, when the first extending direction is X direction, the second extending direction is Y direction. When the first extending direction is Y direction, the second extending direction is X direction.

When the instruction for changing the display magnification is input for one of the plurality of tomographic images 80 extending in the first extending direction, the CPU 41 also synchronously changes the display magnifications of the plurality of tomographic images extending in the second extending direction. As one example, the CPU 41 changes the display magnifications of the plurality of tomographic images 80 extending in the second extending direction while synchronizing the display range in the depth direction of each of the plurality of tomographic images 80 extending in the first extending direction and the display range in the depth direction of each of the plurality of tomographic images 80 extending in the second extending direction with each other. Further, the CPU 41 changes the display magnifications of the plurality of tomographic images 80 extending in the second extending direction while synchronizing the display range in the second extending direction of each of the tomographic images 80 extending in the second extending direction and the display range in the second extending direction of each of the front images 70 with each other.

Next, the CPU 41 determines whether a reset instruction has been input to return back the display magnification and the display position of the image to an initial state for the image (S57). If not input (S57: NO), the process proceeds to S59. When the reset instruction is input (S57: YES), the CPU 41 resets at least one of the display magnifications of the plurality of front and tomographic images 70, 80 and the display position in the display frame of the monitor 47 to the initial state at the time of starting the display (S58). Therefore, the user can return the display magnifications and the display positions of the plurality of front and tomographic images 70, 80 back to the state at the time of starting the display with a simple operation.

If an instruction to terminate the process is not input (S59: NO), the process returns back to S41, and the steps of S41-S59 are repeated. When the termination instruction is input (S59: YES), the image display process is terminated.

According to the second embodiment, the user can appropriately recognize the condition of the living tissue by comparing the plurality of image sets taken from the same subject living tissue. Further, when the display magnification change instruction is input into the monitor 47 for one of the plurality of front and tomographic images 70, 80, not only the display magnification of the image for which the display magnification change instruction is input, but also the display magnifications of other images (the front images 70 and the tomographic images 80) that are displayed on the monitor 47 are changed in accordance with the input instruction. Thus, the user can compare the plurality of images (the front images 70 and the tomographic images 80) more easily and appropriately as compare to individually changing the magnification of each of the plurality of images.

The technology disclosed in the above embodiments is only one example. Accordingly, it is also possible to change the technology exemplified in the above embodiments. First, only a part of the processes exemplified in the above-described embodiment may be executed. Further, the CPU 41 may change, in a region of the three-dimensional image 60, the position at which the tomographic image 80 to be displayed on the monitor 47 is extracted according to the position at which the front image 70 is enlarged. For example, the CPU 41 maintains the extraction position for the tomographic image 80 in the region of the three-dimensional image 60 at a specific position (e.g., the center position) in the display frame of the front image 70 displayed on the monitor 47. In this case, since the relative positional relationship between the acquired front image 70 and the tomographic image 80 is appropriately changed, the condition of the living tissue can be appropriately acquired.

The steps of acquiring images at S2 and S4 in FIG. 6 and S32 and S35 in FIG. 9 are one example of an “image acquisition step”. The steps of displaying an image at S5 in FIG. 6 and S36 in FIG. 9 are one example of an “image display step”. The steps of changing the display magnification of the image at S18-S26 in FIG. 7 and S48-S56 in FIG. 10 are one example of the “magnification synchronization change step”. At S28 of FIG. 7 and S58 of FIG. 10, the steps of resetting the display magnification and display position of the image are one example of a “display reset step”. The steps of changing the display magnification of the image at S16 in FIG. 7 and S46 in FIG. 10 are one example of a “magnification single change step”.

1. A medical image processing method for processing image data of a living tissue, comprising:
acquiring a front image and a tomographic image that are captured from a same living tissue of a same subject, wherein the front image is a two-dimensional image of the living tissue when viewed in a direction along an optical axis of imaging light, and the tomographic image is a two-dimensional image that spreads in a depth direction of the living tissue;
displaying the front image and the tomographic image on a display unit;
specifying one of the front image and the tomographic image that are displayed on the display unit by setting a reference position on the one of the front image and the tomographic image through an operation unit controlled by a user, wherein the reference position serves a reference for enlarging and minimizing an image displayed on the display unit;
changing a display magnification of the specified one of the front image and the tomographic image in response to the user operating the operation unit; and
synchronously changing a display magnification of an other of the front image and the tomographic image in accordance with a change in the display magnification of the specified one of the front image and the tomographic image.

2. A medical image processing device that processes image data of a living tissue, comprising
a control unit that is configured to perform:
an image acquisition step of acquiring a front image and a tomographic image that are captured from a same living tissue of a same subject, wherein the front image is a two-dimensional image of the living tissue when viewed in a direction along an optical axis of imaging light, and the tomographic image is a two-dimensional image that spreads in a depth direction of the living tissue;
an image display step of displaying the front image and the tomographic image on a display unit; and
when an instruction for changing a display magnification of one of the front image and the tomographic image is input into the display unit, a display magnification change step of changing, in accordance with the input instruction, both a display magnification of the one of the front image and the tomographic image for which the instruction was given and a display magnification of an other of the front image and the tomographic image for which the instruction was not given.

3. The medical image processing device according to claim 2, wherein
the front image is an angography front image generated by processing a plurality of OCT signals acquired by an OCT device at different timings at a same position of the living tissue.

4. The medical image processing device according to claim 2, wherein
the tomographic image is captured by an OCT device that is configured to capture an image of a living tissue using a principle of optical coherence tomography, and
information of motion contrast data generated by processing a plurality of OCT signals that are acquired by the OCT device at different timings at a same position of the living tissue is superimposed on the tomographic image.

5. The medical image processing device according to claim 2, wherein
at the magnification synchronization change step, the control unit is further configured to change both the display magnification of the front image and the display magnification of the tomographic image while synchronizing a display range in an extending direction, which is a direction perpendicular to the depth direction, for the tomographic image in the display unit and a display range in the extending direction for the front image in the display unit with each other.

6. The medical image processing device according to claim 2, wherein
the control unit is further configured to:
acquire, at the image acquisition step, the front image, a first tomographic image that spreads in a first extending direction that is one of directions perpendicularly intersecting the depth direction, and a second tomographic image that spreads in a second extending direction that perpendicularly intersects the depth direction and is different from the first extending direction;
display, at the image display step, the front image, the first tomographic image, and the second tomographic image on the display unit; and
when an instruction for changing the display magnification of one of the front image, the first tomographic image, and the second tomographic image that are displayed on the display unit is input, synchronously change, at the magnification synchronization change step, the display magnification of the front image, the display magnification of the first tomographic image, and the display magnification of the second tomographic image.

7. The medical image processing device according to claim 6, wherein
the control unit is further configured to perform:
a first reference position setting step of setting a first reference position that serves as a reference for expanding and minimizing the first tomographic image in the first extending direction; and
a second reference position setting step of setting a second reference position that serves as a reference for expanding and minimizing the second tomographic image in the second extending direction, and
at the magnification synchronization change step, the control unit is further configured to synchronously change the display magnification of the first tomographic image and the display magnification of the second tomographic image by setting the first reference position as the reference for expanding and minimizing the first tomographic image and setting the second reference position as the reference for expanding and minimizing the second tomographic image.

8. The medical image processing device according to claim 7, wherein
the control unit is further configured to perform a reference position display step of displaying the first reference position and the second reference position on the front image displayed on the display unit.

9. The medical image processing device according to claim 2, wherein
at the magnification synchronization change step, the control unit is further configured to:
receive an instruction for specifying a reference position in a display range of the front image or a display range of the tomographic image, wherein the reference position serves as a reference for enlarging and minimizing; and
perform an enlarging process or a minimizing process on at least one of the front image and the tomographic image based on the specified reference position.

10. The medical image processing device according to claim 2, wherein
when an instruction for returning at least one of a display magnification and a display position to an initial state is received, the control unit is further configured to perform a display reset step of resetting at least one of (i) the display magnification of the front image and the display magnification of the tomographic image and (ii) the display position for displaying within a display frame of the display unit to the initial state of starting display.

11. The medical image processing device according to claim 2, wherein
the control unit is further configured to perform, when an instruction for changing the display magnification of one of the front image and the tomographic image is input into the display unit, a magnification single change step of enlarging or minimizing, in accordance with the input instruction, only the display magnification of the one of the front image and the tomographic image for which the display magnification change instruction was given, and
the control unit is further configured to selectively perform either the magnification synchronization change step or the magnification single change step in accordance with an instruction input by a user.

12. The medical image processing device according to claim 2, wherein
the control unit is further configured to:
acquire, at the image acquisition step, a plurality of image sets each of which is a set of the front image and the tomographic image that are captured from a same living tissue of a same subject;
display, at the image display step, the plurality of image sets on the display unit; and
when an instruction for changing the display magnification of one of a plurality of front images and a plurality of tomographic images included in the plurality of image sets is input into the display unit at the magnification synchronization change step, synchronously change the display magnifications of all the plurality of front images and the display magnifications of all the plurality of tomographic images included in the plurality of image sets in accordance with the input instruction.

13. The medical image processing device according to claim 12, wherein
the control unit is further configured to:
align, at the image display step, the plurality of front images included in the plurality of image sets with each other; and
display, on the display unit, the plurality of front images that have been aligned.

14. The medical image processing device according to claim 12, wherein
the control unit is further configured to, at the magnification synchronization change step:
set a reference position for enlarging and minimizing at a same position in a display range of each of the plurality of front images;
perform an enlarging process or a minimizing process on the plurality of front images based on the set reference position;
set a reference position for enlarging and minimizing at a same position in a display range of each of the plurality of tomographic images; and
perform an enlarging process or a minimizing process on the plurality of tomographic images based on the set reference position.

15. A non-transitory, computer readable, storage medium storing a medical image processing program executed by a medical image processing device that processes image data of a living tissue, the medical image processing program, when executed by a control unit of the medical image processing device, causing the control unit to perform:
an image acquisition step of acquiring a front image and a tomographic image that are captured from a same living tissue of a same subject, wherein the front image is a two-dimensional image of the living tissue when viewed in a direction along an optical axis of imaging light, and the tomographic image is a two-dimensional image that spreads in a depth direction of the living tissue;
an image display step of displaying the front image and the tomographic image on a display unit; and
when an instruction for changing a display magnification of one of the front image and the tomographic image is input into the display unit, a display magnification change step of changing, in accordance with the input instruction, both a display magnification of the one of the front image and the tomographic image for which the instruction was given and a display magnification of an other of the front image and the tomographic image for which the instruction was not given.
